(19) United States
(12) Patent Application Publication
(10) Pub. No.: US 2026/0240533 A1
(43) Pub. Date: Aug. 20, 2026

(54) JOINT DISTRACTION LEVER AND ORTHOPEDIC IMPLANTS WITH INTEGRATED STRAIN GAUGES

(71) Applicant: MAKO Surgical Corp., Weston, FL (US)

(72) Inventors: Peter Ebbitt, Weston, FL (US); David Bowling, Los Ranchos, NM (US); Snehal Kasodekar, Weston, FL (US); Remy Aubrey, Nelson (NZ)

(73) Assignee: MAKO Surgical Corp., Weston, FL (US)

(21) Appl. No.: 19/541,159

(22) Filed: Feb. 16, 2026

Related U.S. Application Data
(60) Provisional application No. 63/759,648, filed on Feb. 18, 2025.
(60) Provisional application No. 63/809,627, filed on May 21, 2025.

(51) Int. Cl. A61B 17/02; A61B 17/00; A61B 17/56; A61B 34/10; A61B 34/30; A61B 90/00

(52) U.S. Cl. CPC A61B 17/025; A61B 17/56; A61B 34/10; A61B 34/30; A61B 90/06; A61B 2017/00022; A61B 2017/00526; A61B 2017/0268; A61B 2017/564; A61B 2090/064

(57) ABSTRACT

A joint distraction lever comprising a lever body having a distal end and a proximal end opposite the distal end, the distal end having a handle portion and the proximal end having a working portion configured to be positioned between a first bone and a second bone of a joint. The joint distraction lever further comprising a strain gauge integrated into the lever body and positioned between the handle portion and the working portion, the stain gauge configured to measure a strain on the lever body indicative of a distraction force applied at the distal end during a distraction procedure.

### CROSS-REFERENCE TO RELATED PATENT APPLICATIONS

This application claims the benefit of and priority to U.S. Provisional Application No. 63/759,648, filed on Feb. 18, 2025, and U.S. Provisional Application No. 63/809,627, filed on May 21, 2025, the entire disclosures of which are hereby incorporated by reference herein.

### BACKGROUND

The present disclosure relates disclosure relates generally to surgical tools for use during planning and preparation of a joint replacement procedure, and more particularly to a joint gap balancing lever for use during joint distraction.

Over time, as a result of disease, injury, or longevity of use, bones of a joint may degenerate, resulting in pain and diminished functionality. To reduce pain and restore functionality, a joint replacement procedure may be necessary. Examples of such procedures may be total or partial knee arthroplasty, total hip arthroplasty, or knee or hip resurfacing. In these procedures, portions of a patient's joint are replaced with artificial components. Particularly, a surgeon uses a surgical cutting tool to remove portions of bone to prepare the bone to receive a prosthetic device. Prior to resection of the bone, the surgeon plans bone preparation specific to the patient's anatomy, size, current state of the target joint, and several other factors in order to determine the portions of the bone that will be removed and replaced by one or more prosthetic components, as well as to determine proper positioning of the one or more prosthetic components.

One step of surgical planning for a knee arthroplasty, partial knee resurfacing procedure, or other knee repair or reconstruction procedures involves a knee joint distraction, that is, forced separation of the distal femur from the proximal tibia. For partial knee resurfacing or total knee arthroplasty, for example, this may be intended to correct knee joint deformity and cause proper re-tensioning of the ligaments of the knee to determine a desired, post-procedure joint construction. In one exemplary method, prior to resection and prior to a creating a final implant plan, the knee joint deformity is corrected at multiple flexion positions or flexion angles by distracting the joint. An instantaneous six degree-of-freedom (DOF) position (i.e., the pose) of the femur with respect to the six DOF position of the tibia is captured at each of the multiple flexion positions.

For example, a common flexion position is near full extension where the surgeon applies a valgus torque to the tibia when the leg is at approximately 5-10 degrees of flexion. The valgus torque corrects the limb alignment deformity and returns the ligaments to a proper tension state. Another common flexion position is 90 degrees flexion. With these two poses, the knee joint is in the desired post-resection final position. After collection of poses, bone resection, implant positioning, and implant characteristics are planned so as to maintain this relative alignment by making the femoral and tibial components contact (or be slightly gapped to allow for some laxity). Once the bone is resected at this desired plan and the trials and/or implants are secured to the bone, the leg will then be in the pre-resected posed positions.

A first technique currently used to apply a joint distraction force includes manually applying a valgus torque (for a varus knee) to the tibia portion of a patient's leg to pivot the knee joint about the contralateral compartment (lateral compartment for a varus knee). Another technique includes applying a distraction force using a common surgical osteotome by levering the osteotome off the front of the tibia and lifting the femur vertically. Similarly, joint distraction may be performed by placing shim-like spoons or gap sticks between the femur and tibia, or by using laminar spreaders to create the distance between the femur and the tibia.

However, for each of these techniques, the “proper” joint distraction force is subjective, varies from surgeon-to-surgeon, and is difficult for surgeons to learn. In addition, for the first technique described above, applying a valgus torque to the tibia for any pose after 30 degrees flexion is extremely difficult because the femur tends to rotate about the femoral head.

Other types of distractors include spring-based, electromechanical, or hydraulic opposing plate spreaders. However, these tend to be large and complex, and due to their size generally require at least some bone to be removed first (provisional resection) to accommodate the device's opposing plates. Yet another device is a force sensing shim. However, like the shim-like gap sticks, this device generally requires iteratively inserting the device into the joint with various thicknesses until the desired force is achieved, making it time consuming and cumbersome.

### SUMMARY

At least one embodiment relates to a joint distract lever. The joint distraction lever comprises a lever body having a distal end and a proximal end opposite the distal end. The distal end includes a handle portion and the proximal end includes a working portion configured to be positioned between a first bone and a second bone of a joint. The joint distraction lever further comprises a strain gauge integrated into the lever body and positioned between the handle portion and the working portion, where the stain gauge configured to measure a strain on the lever body indicative of a distraction force applied at the distal end during a distraction procedure.

Another embodiment relates to a surgical system for performing a joint distraction. The system includes a joint distraction lever comprising a lever body having a distal end and a proximal end opposite the distal end. The distal end includes a handle portion and the proximal end includes a working portion configured to be positioned between a first bone and a second bone of a joint. The joint distraction lever further comprises a strain gauge integrated into the lever body and positioned between the handle portion and the working portion, where the stain gauge configured to measure a strain on the lever body indicative of a distraction force applied at the distal end during a distraction procedure. The surgical system also comprises a computing system configured to provide a surgical plan, and a robotic device coupled with the joint distraction lever, where the robotic device configured to apply a force to the handle portion in accordance with the surgical plan to cause the working portion to apply the distraction force.

Another embodiment relates to a method for applying a distraction force during a distraction procedure. The method includes providing a joint distraction lever comprising a lever body having a distal end and a proximal end opposite the distal end. The distal end includes a handle portion and the proximal end includes a working portion configured to be positioned between a first bone and a second bone of a joint. The joint distraction lever further comprises a strain gauge integrated into the lever body and positioned between the handle portion and the working portion, where the stain gauge configured to measure a strain on the lever body indicative of a distraction force applied at the distal end during the distraction procedure. The method also includes inserting the joint distraction lever into a space between the first bone and the second bone, and applying a force to the handle portion such that the working portion applies the distraction force to the joint. The method further includes measuring, via the strain gauge, a force value associated with the distraction force, and outputting the measured force value associated with the distraction force.

Another embodiment relates to an implant. The implant includes an implant body having a first end, a second end opposite the first end, and a central region between the first end and the second end. The central region is configured to be positioned between a first bone portion and a second bone portion. A strain gauge is integrated into the implant body and positioned in the central region between the first end and the second end. The strain gauge is configured to measure a strain on the central region indicative of a force experienced by at least one of the central region, the first end, or the second end. The implant may include compression aperture in the central region configured to facilitate measurement of the strain by the strain gauge.

In some embodiments, the implant body is a plate, the implant body further comprising a plurality of apertures extending through the central region of the implant body. In some embodiments, the strain gauge is positioned between two apertures of the plurality of apertures. In other embodiments, the strain gauge is positioned between at least one aperture of the plurality of apertures and an edge of the implant body.

In some embodiments, the first end is a head, the second end is a stem, and the central region is a neck, where the implant further comprises a compression aperture extending through the neck and positioned adjacent to the strain gauge, where the compression aperture configured to permit at least a portion of the neck to bias in response to the force experienced by at least one of the head, the stem, or the neck.

In some embodiments, the implant body is a cage base defining a central cavity, the cage base having a first opening at a first lateral side and a second opening at a top side. In some embodiments, the strain gauge is integrated with a surface defining a lower boundary of the first opening at the first lateral side of the cage base. In other embodiments, the strain gauge is integrated with a surface at the top side, the strain gage positioned between the second opening and an edge of the cage base at the first lateral side. In other embodiments, the cage base includes a first base member coupled with a second base member via an expansion member, where the first base member and the second base member are configured to move relative to one another between an expanded configuration and a retracted configuration, and where the strain gauge is coupled with the expansion member.

In some embodiments, the first end is a head, the second end is a shaft having a threaded exterior, and the central region is a neck, and where the implant further comprises a compression aperture extending through the neck and positioned adjacent to the strain gauge.

In some embodiments, the implant is a stabilization rod configured to couple a bone screw, and where the implant further comprises a compression aperture extending through the implant body and positioned adjacent to the strain gauge.

This summary is illustrative only and is not intended to be in any way limiting. Other aspects, features, and advantages of the devices or processes described herein will become apparent in the detailed description set forth herein, taken in conjunction with the accompanying figures, wherein like reference numerals refer to like elements.

### BRIEF DESCRIPTION OF THE FIGURES

The disclosure will become more fully understood from the following detailed description, taken in conjunction with the accompanying figures, wherein like reference numerals refer to like elements, in which:

FIG. 1 is a perspective view of a surgical system, according to an exemplary embodiment.

FIG. 2 is a top perspective view of a joint distraction lever of the surgical system of FIG. 1, according to an exemplary embodiment.

FIG. 3 is a bottom perspective view of a joint distraction lever of the surgical system of FIG. 1, according to an exemplary embodiment.

FIG. 4 is a side view of a joint distraction of a patient's knee using a joint distraction lever of the surgical system of FIG. 1, according to an exemplary embodiment.

FIG. 5 is a view of a force profile of a joint distraction lever of the surgical system of FIG. 1 during a joint distraction, according to an exemplary embodiment.

FIG. 6 is one embodiment of a method of performing a joint distraction using a joint distraction lever of the surgical system of FIG. 1, according to an exemplary embodiment.

FIG. 7 is one embodiment of a method of performing a gap balancing using a joint distraction lever of the surgical system of FIG. 1, according to an exemplary embodiment.

FIG. 8 is one embodiment of a top perspective view of a plate that can be used with the surgical system of FIG. 1, according to an exemplary embodiment.

FIG. 9 is one embodiment of a top perspective view of a plate that can be used with the surgical system of FIG. 1, according to an exemplary embodiment.

FIG. 10 is one embodiment of a top perspective view of a plate that can be used with the surgical system of FIG. 1, according to an exemplary embodiment.

FIG. 11 is one embodiment of a top perspective view of a plate that can be used with the surgical system of FIG. 1, according to an exemplary embodiment.

FIG. 12 is one embodiment of a perspective view of an implant that can be used with the surgical system of FIG. 1, according to an exemplary embodiment.

FIG. 13 is one embodiment of a top perspective view of a cage that can be used with the surgical system of FIG. 1, according to an exemplary embodiment.

FIG. 14 is one embodiment of a top perspective view of a cage that can be used with the surgical system of FIG. 1, according to an exemplary embodiment.

FIG. 15 is one embodiment of a top perspective view of a cage that can be used with the surgical system of FIG. 1, according to an exemplary embodiment.

FIG. 16 is one embodiment of a top perspective view of a cage that can be used with the surgical system of FIG. 1, according to an exemplary embodiment.

FIG. 17 is one embodiment of a perspective view of a stabilization system that can be used with the surgical system of FIG. 1, according to an exemplary embodiment.

FIG. 18 is one embodiment of an implantation diagram of a distraction device that can be used with the surgical system of FIG. 1, according to an exemplary embodiment.

FIG. 19 is one embodiment of an implantation diagram of a distraction device that can be used with the surgical system of FIG. 1, according to an exemplary embodiment.

FIG. 20 is one embodiment of an implantation diagram of a distraction device that can be used with the surgical system of FIG. 1, according to an exemplary embodiment.

FIG. 21 is one embodiment of an implantation diagram of an implant that can be used with the surgical system of FIG. 1, according to an exemplary embodiment.

FIG. 22 is one embodiment of a system that can be used with the surgical system of FIG. 1, according to an exemplary embodiment.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

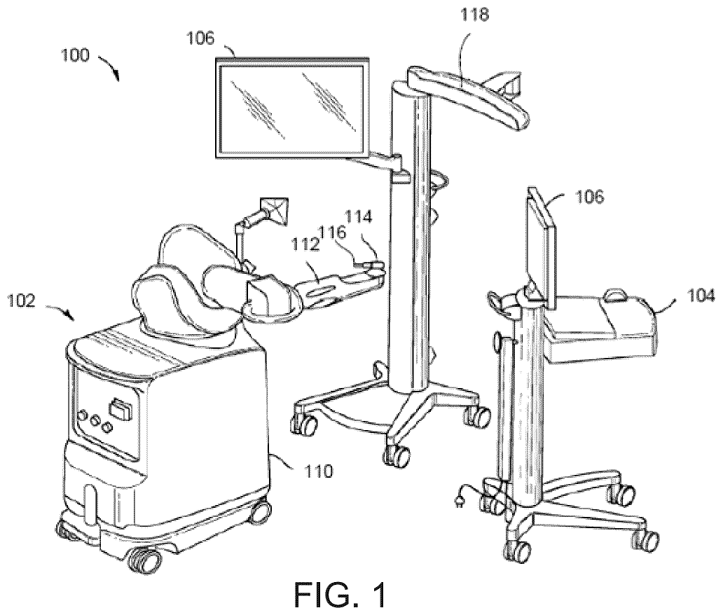

Referring generally to the Figures, exemplary surgical systems, apparatuses, and methods are disclosed. As shown in FIG. 1, an exemplary surgical system 100 is shown. The surgical system 100 may be a computer-assisted surgical (CAS) system, a computer-implemented surgical system, and/or any other suitable surgical system. The surgical system 100 may be configured to perform a variety of surgical procedures (e.g., an orthopedic procedure, a total or partial knee arthroplasty procedure, a total or partial knee replacement procedure, a total or partial hip arthroplasty, a total or partial hip replacement procedure, a shoulder replacement operation, a knee, hip, or shoulder revision procedure, a hip scoping procedure, a vertebral procedure, a foot or ankle procedure, a hand or wrist procedure, a neurosurgical procedure, a trauma procedure, etc.). It should be understood that the systems described herein (e.g., the surgical system 100, a robotic system, a computing system, etc.), as well as the concepts and processes described herein, may be applicable to any suitable type of surgical procedure (e.g., joint replacement including revision procedures, trauma procedures as for fracture and break repair, sports medicine procedures as for ligament repair, etc.). In some embodiments, the surgical system 100 includes additional, fewer, and/or different components (e.g., a surgical bed, a surgical lighting device, a tracking system, a tracking device, etc.).

As shown in FIG. 1, the surgical system 100 includes a robotic system 102, a computing system 104, and at least one display device, shown as display device 106. The robotic system 102 may include a base 110, an arm 112, a force system, and a controller. In some embodiments, the arm 112 is an articulated arm; however, in other embodiments, the arm 112 is another suitable type of arm (e.g., a telescoping arm, etc.).

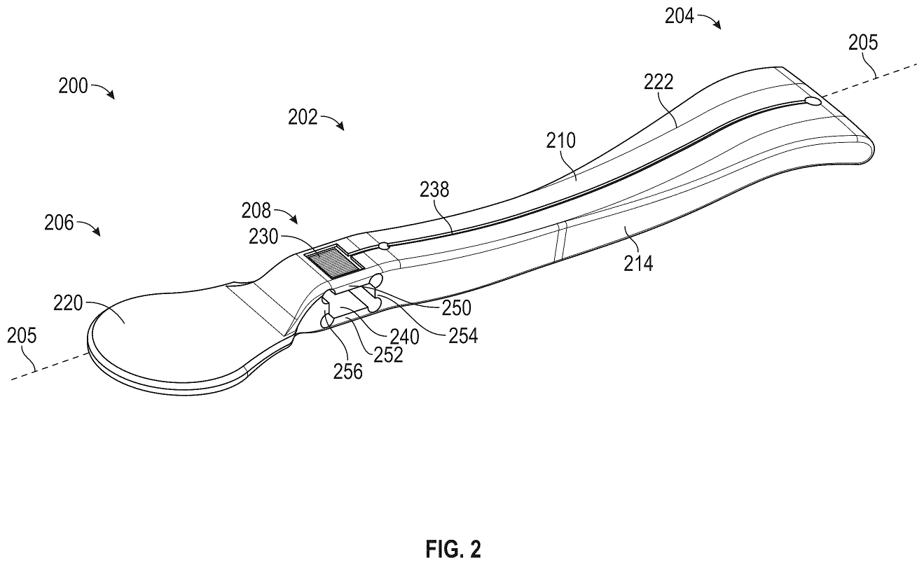

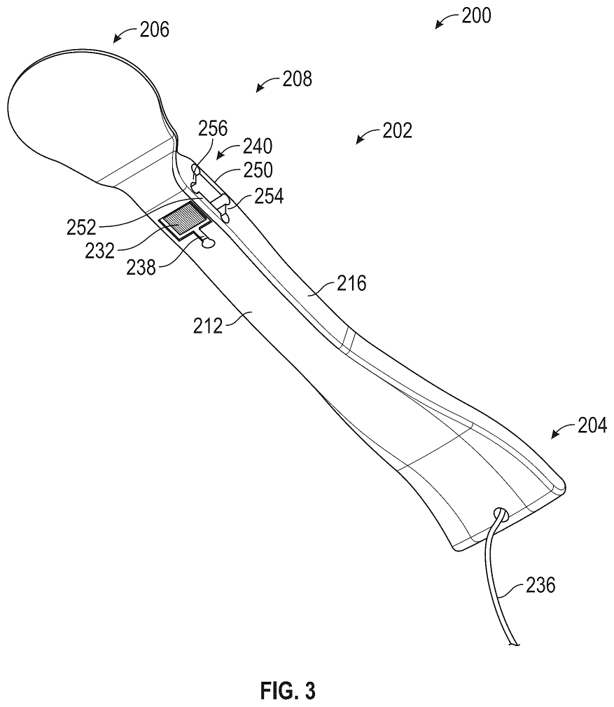

The surgical system 100 may further include a tool, shown as surgical tool 114 (e.g., in at least FIG. 1). In some embodiments, and as described herein, the surgical tool 114 is a joint distraction device, such as a joint distraction lever, a joint gap balancing device, such as a joint gap balancing lever, and/or another suitable surgical tool described herein (e.g., another suitable distraction lever-type and/or gap-balancing device, etc.). In some embodiments, the surgical system 100 includes a plurality of surgical tools 114, which may be removably coupled to the robotic system 102 (e.g., the arm 112, etc.). For example, the surgical system 100 may include a first surgical tool 114 (e.g., a joint distraction tool as shown in at least FIGS. 2-3) and a second surgical tool (e.g., a cutting tool as shown in at least FIG. 1), which may be removably coupled to the robotic system 102 (e.g., throughout a surgical procedure).

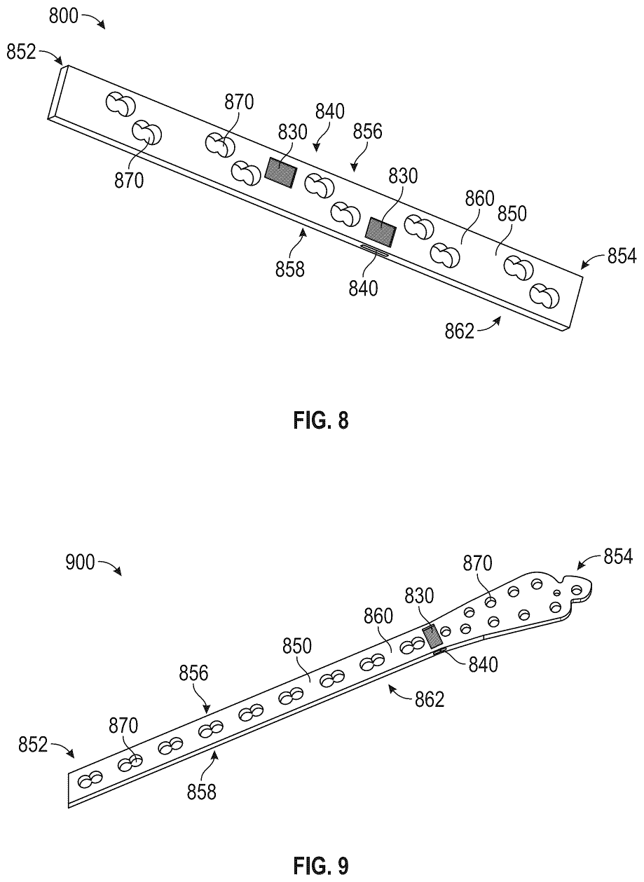

In other embodiments, and as will be discussed herein, the surgical system 100 may include a first surgical tool 114 (e.g., a joint distraction tool as shown in at least FIGS. 2-3), and one or more devices (e.g., an implant, an implantable device, a prosthetic device, for example as described with reference to FIGS. 8-21), which may be removably coupled to the robotic system 102 (e.g., throughout a surgical procedure, etc.) and/or implanted, inserted, or coupled into/to a patient. In this regard, the surgical system 100 may include one or more surgical tools for performing one or more operations (e.g., a joint distraction tool for performing a joint distraction and/or gap balancing operation, etc.), and/or one or more devices (e.g., an implant or prosthetic device, etc.), for example for monitoring and/or analysis of one or more characteristics or conditions (e.g., a condition, performance, and/or function of the device and/or anatomical features, etc.), as described herein.

As shown in FIG. 1, the surgical tool 114 may include an end effector (e.g., coupled to the arm 112), shown as cutting tool 116. The cutting tool 116 may include an operating member or cutting feature, such as a saw, reamer, burr, or other suitable operating member (e.g., an ultrasonic cutting tool, a water jet, a vibrating tool, etc.). In some embodiments, the robotic system 102 is used in an interactive manner (e.g., by a healthcare provider, a surgeon, a user, an operator, etc.) to implement or perform a surgical procedure or operation. For example, the robotic system 102 (e.g., the arm 112, the surgical tool 114, etc.) may be manipulated, such that the arm 112 and/or the surgical tool 114 is/are manipulated (e.g., moved, repositioned, etc.) to implement or perform the surgical procedure.

As described herein, the computing system 104 may include hardware and/or software components (e.g., one or more processors, one or more memory devices, computer-readable instructions stored on one or more memory devices, etc.), for example to implement or perform the one or more operations described herein. For example, the computing system 104 may be configured to determine a position and/or orientation associated with one or more objects (e.g., features, devices, components, etc.) within a surgical environment. The computing system 104 may be configured to determine a position and/or orientation associated with one or more objects, for example to implement or perform a surgical procedure and/or plan a surgical procedure. In some embodiments, the computing system 104 may include surgical planning and/or surgical assistance software, which may be used to implement and/or perform a surgical procedure. For example, the computing system 104 may be configured to store one or more images, videos, models (e.g., a three-dimensional model, a virtual model, etc.), which may be used to implement and/or plan a surgical procedure, and/or may be displayed on the display device 106.

In an exemplary embodiment, the computing system 104 is communicably coupled with the robotic system 102 and/or the display device 106. It should be understood that in some embodiments one or more components of the surgical system 100 is/are combined and/or implemented in one or more devices. For example, in some embodiments the robotic system 102, the computing system 104, and/or the display device 106 is/are implemented in a single component or device (e.g., the robotic system 102).

The surgical system 100 may be configured to determine a pose (e.g., a position, orientation, a position and orientation, etc.) of one or more objects in a surgical environment. For example, the robotic system 102 and/or the computing system 104 may be configured to determine a pose of one or more objects in a space (e.g., a surgical environment, etc.). Further, the surgical system 100 may be configured to establish one or more boundaries (e.g., a haptic region, a haptic restraint, etc.) in a surgical environment. For example, the robotic system 102 and/or the computing system 104 may be configured to determine one or more virtual boundaries using one or more objects in space (e.g., a surgical environment, etc.). In addition, the surgical system 100 may be configured to establish a coordinate frame of reference in a surgical environment. For example, the robotic system 102 and/or the computing system 104 may be configured to establish a coordinate frame of reference using one or more objects in space (e.g., a surgical environment, etc.).

As discussed herein, in some embodiments the robotic system 102 (e.g., the arm 112, etc.) and/or the computing system 104 is configured to determine a pose of one or more objects in a space, for example a component of the surgical system 100 (e.g., the surgical tool 114, etc.). As described herein, an object may include, for example, a tool, an instrument, a patient anatomy, an implant, a prosthetic device, a point in space, a surface (e.g., of a patient anatomy, an implant, a prosthetic device, etc.), and/or one or more components of the surgical system 100. The robotic system 102 and/or the computing system 104 may include sensors, joint actuators, joint controllers, encoders (e.g., at joints of the arm 112), and/or other mechanical and/or computing components (e.g., an optical tracking system, or a tracking system 118, etc.), configured to determine (e.g., establish, identify, etc.) a pose of the one or more objects. For example, the robotic system 102 may also include the tracking system 118, which may be used to detect (e.g., optically, etc.) a pose of the surgical tool 114, either directly or indirectly, for example by detecting a pose of one or more components (e.g., an array, etc.) of the base 110, the arm 112, and/or the surgical tool 114.

Further, in some embodiments the surgical system 100 is also configured to register coordinates in a space or environment. For example, the robotic system 102 and/or the computing system 104 may be configured to register (e.g., map, associate, etc.) coordinates in a space or environment, for example to determine a spatial alignment or correspondence between one or more objects in the space or environment (e.g., determine or establish a coordinate frame, for example through a coordinate transformation process, etc.). Objects in physical space may be registered to any suitable coordinate system, for example a coordinate system being used by the robotic system 102 (e.g., a controller, etc.) and/or the computing system 104 (e.g., a controller, processor, memory device, etc.), as discussed herein. In some embodiments, the surgical system 100 (e.g., object data from the robotic system 102, the computing system 104, etc.) uses the object data to establish a coordinate frame of reference (e.g., relative to the object, relative to one or more objects, etc.). For example, the surgical system 100 may establish a coordinate frame of reference using one or more identified objects (e.g., an identified object defining a center of the coordinate frame, a plurality of identified objects defining one or more axis or planes of the coordinate frame, a virtual point spaced a distance from an identified object, etc.).

In some embodiments, the surgical system 100 uses object data to associate a physical anatomy of a patient (e.g., a patient's tibia, etc.) with a coordinate frame of reference. For example, the surgical system 100 may establish a coordinate frame of reference using a first identified object (e.g., an object defining a center of the coordinate frame, etc.), and associate a physical anatomy of a patient using a second identified object (e.g., a patient's tibia using an identification of the lateral condyle, the medial condyle, the tibial tuberosity, etc.). In other embodiments, the surgical system 100 also uses object data to associate a physical anatomy of patient (e.g., a patient's tibia, etc.) with a representation of the anatomy (e.g., an image of the physical anatomy, a virtual model of the physical anatomy, etc.), for example by performing a best fit of points collected by tracking the pose of an object (e.g., cutting tool, probe, etc.) by the surgical system 100 with a surface of a model of the patient's anatomy (e.g., based on pre-operative CT or other imaging). In some embodiments, the surgical system 100 is configured to establish a coordinate frame of reference, associate a physical anatomy of a patient relative to a coordinate frame, associate a representation of the anatomy relative to a coordinate frame, and/or any combination thereof (e.g., using object data). In this regard, using the object data (e.g., an identified object, a tracked object, etc.), the surgical system 100 may determine one or more spatial relationships between a coordinate frame, an anatomy of a patient, a representation of an anatomy of a patient, and/or a combination thereof.

In an exemplary embodiment, registration includes any suitable registration technique. For example, the registration technique may include physical-space registration (e.g., where a patient's actual anatomy is registered relative to a coordinate frame), image-to-image registration (e.g., monomodal registration where images of the same type or modality, such as fluoroscopic images or MR images, are registered and/or multimodal registration where images of different types or modalities, such as MRI and CT, are registered), image-to-physical space registration (e.g., image-to-patient registration where a digital data set of a patient's anatomy obtained by conventional imaging techniques is registered with the patient's actual anatomy), combined image-to-image and image-to-physical-space registration (e.g., registration of preoperative CT and MRI images to an intraoperative scene), and/or registration using a video camera, ultrasound, and/or another suitable system or device (e.g., a tracking system, etc.).

The surgical system 100 (e.g., the robotic system 102, the computing system 104, etc.) may also include a coordinate transform process for mapping (or transforming) coordinates in one space to those in another to achieve spatial alignment or correspondence. For example, the surgical system 100 may use the coordinate transform process to map positions of identified objects (e.g., an identified object for registration, an identified object for coordinate frame registration, an identified object on a patient anatomy, an identified object to determine a virtual boundary, etc.) into a coordinate system used by a process running on the computer of a haptic device and/or a surgical controller. The coordinate transform process may include any suitable transformation technique, such as, for example, rigid-body transformation, non-rigid transformation, affine transformation, and the like. In some embodiments, a camera and/or a scan of patient anatomy can be used to obtain a model and/or register the model. For example, an initial 3D model can be created and registered. In some embodiments, a video camera can be used to register a 3D model corresponding to a CT scan. According to some embodiments, a video camera, ultrasound, and/or another suitable system or device (e.g., a tracking system, etc.) can be used for both initial model creation and/or registration.

As discussed above, in some embodiments the surgical system 100 is also configured to determine one or more boundaries in space (e.g., a surgical environment, etc.). For example, the robotic system 102 and/or the computing system 104 may be configured identify an object (e.g., an object in space, an anatomy of a patient, etc.) with respect to a coordinate frame of reference, for example to determine a pose (e.g., a position, etc.) of the identified object (e.g., relative to the frame of reference). In some embodiments, the robotic system 102 (e.g., via a pose of the surgical tool 114, etc.) is configured to identify an object (or objects) with respect to a coordinate frame of reference, for example to establish one or more virtual boundaries in space. As described herein, the one or more virtual boundaries may provide a guide or restraint (e.g., a boundary, a limit, etc.), for example to control or guide manipulation of a surgical tool (e.g., the surgical tool 114, etc.). In this regard, the surgical system 100 (e.g., the robotic system 102, the computing system 104, the force system, etc.) may be implemented with one or more virtual boundaries, for example to control or guide manipulation of a surgical tool during a surgical operation or procedure.

In some embodiments, the surgical system 100 (e.g., the force system, controller, etc.) is configured to provide a restraint guide via control or guidance to the surgeon during manipulation of the surgical tool 114. The force system (e.g., motors operating joints of the arm 112) may be configured to provide at least some force to the surgical tool 114 via the arm 112, and a controller may be programmed to generate control signals for controlling the force system. In some embodiments, the force system includes actuators and a back-driveable transmission that provide haptic (or force) feedback to constrain or inhibit the surgeon from moving the surgical tool beyond predefined haptic boundaries defined by haptic objects as described, for example, in U.S. Pat. No. 8,010,180 and/or U.S. patent application Ser. No. 12/654,519 (U.S. Patent Application Pub. No. 2010/0170362), filed Dec. 22, 2009, each of which is hereby incorporated by reference herein in its entirety. As described herein, the force system and/or the controller may be housed within the robotic system 102 and/or the computing system 104. In some embodiments, restraint or guidance is provided though a handheld manipulator or handheld robotic device, such as described in U.S. Pat. No. 9,399,298 entitled “Apparatus and Method for Providing an Adjustable Positive Stop in Space,” U.S. Pat. No. 9,060,794 entitled “System and Method for Robotic Surgery,” and U.S. Patent Publication No. 2013/0060278 entitled “Surgical instrument including housing, a cutting accessory that extends from the housing and actuators that establish the position of the cutting accessory relative to the housing,” each of which is incorporated herein by reference in its entirety.

In some embodiments, the surgical system 100 is configured to continually determine (e.g., track, etc.) a pose of any relevant and/or identified object (e.g., an anatomy of a patient, a prosthetic, a boundary, etc.). For example, the surgical system 100 (e.g., the robotic system 102, etc.) may include non-mechanical tracking components, mechanical tracking components, and/or any combination of non-mechanical and mechanical tracking components suitable for use in a surgical environment. The non-mechanical tracking components may include virtual, optical (or visual), magnetic, radio, or acoustic tracking components. Such components are configured to be associated with (e.g., appointed to, assigned to, etc.) an object to be tracked (e.g., a point in space, an anatomical feature, an anatomical surface, etc.) and/or may be an inherent component of the object to be tracked (e.g., an identifiable anatomical feature, an identifiable prosthetic feature, etc.). For example, a trackable element (e.g., a virtual trackable element, etc.) may include an array of objects identified on a surface of a patient's anatomy (e.g., via a pose of the surgical tool 114), which may define a geometric arrangement and/or a geometric relationship of the trackable element relative a patient's anatomy. In some embodiments, trackable elements (e.g., a virtual trackable element) include objects identified on a patient's anatomy (e.g., via a pose of the surgical tool 114), which may define a known geometric arrangement of a particular patient's anatomy (e.g., relative to a defined coordinate frame, etc.). Thus, the surgical system 100 can recognize a particular identified object (e.g., a marked or tracked object, etc.), at least in part, from the geometry of the markers, an orientation of the axis, and/or a location of the endpoint within a frame of reference deduced from positions of the markers.

As described herein, the trackable marker (e.g., identified objects, etc.) may include any known marker, for example intrinsic features of an identified object (e.g., a tracked object). For example, the markers may include intrinsic features that are salient and/or accurately locatable portions of objects sufficiently defined and/or identifiable to function as recognizable markers (e.g., anatomical landmarks, outlines of anatomical structures, shapes, outlines of and/or on anatomical features, etc.). In some embodiments, the markers are extrinsic markers (e.g., markers affixed to skin, markers implanted in bone, fiducial arrays, stereotactic frames, etc.) designed to be accurately detectable by the surgical system 100. According to an exemplary embodiment, the markers (e.g., identified objects) are identified (e.g., determined, tracked, located, etc.) using any suitable detection method. For example, the markers may be identified (e.g., determined, established, etc.) via a pose of one or more identified objects (e.g., an object identified by the robotic system 102 via a pose of the surgical tool 114, etc.). In some embodiments, the markers are identified using a tracking device or system, for example the tracking system 118, which may be used to detect a pose of the markers (e.g., directly, indirectly via a pose of one or more components, etc.). In some embodiments, the markers are identified using another suitable system or device (e.g., a detection device, a tracking system, an optical tracking system, a magnetic tracking system, etc.).

Referring now to FIGS. 2-4, a distraction lever 200 is shown, according to an exemplary embodiment. According to an exemplary embodiment, the distraction lever 200 is a component of the surgical system 100 of FIG. 1. For example, the distraction lever 200 may be configured to removably couple the robotic system 102 (e.g., the arm 112, etc.), for example such that the surgical system 100 (e.g., the robotic system 102, the arm 112, etc.) may control and/or guide movement of the distraction lever 200, as described herein. In this sense, in some embodiments the distraction lever 200 is the surgical tool 114 of FIG. 1. In other embodiments, the distraction lever 200 is used in addition to, in combination with, and/or along with one or more components of the surgical system 100. For example, a user or operator may manually control and/or guide use of the distraction lever 200, for example in combination with operation of the surgical tool 114 (e.g., the robotic system 102, the arm 112, etc.), as described herein.

According to an exemplary embodiment, the distraction lever 200 is a joint distraction device, such as a joint distraction lever, a joint gap balancing device, such as a joint gap balancing lever, and/or another suitable surgical distraction lever-type and/or gap-balancing device. Though the present disclosure will refer to the joint distraction device as the distraction lever 200, it is to be understood that the features disclosed herein may be used with and/or provided in a variety of distraction lever-type and gap-balancing devices, which are considered to be within the scope of the present disclosure. According to an exemplary embodiment, and as will be described herein, the distraction lever 200 is configured to measure and/or provide an output related to a force (e.g., a distraction force, torque, etc.) applied to one or more bones of a joint (e.g., during a distraction procedure). In an exemplary embodiment, the distraction force is determined (e.g., calculated, etc.) using measurements from one or more measurement devices (e.g., sensors, strain gauges, etc.), for example to correlate one or more measured forces (e.g., at the measurement device) with one or more applied forces (e.g., at one or more bones of the joint).

As shown in FIGS. 2-4, the distraction lever 200 includes a body 202 having a first end, shown as a proximal end 204, and a second end opposite the first end, shown as distal end 206. The body 202 may extend from the proximal end 204 to the distal end 206. The body 202 may define an axis 205. For example, the body 202 may extend from the proximal end 204 to the distal end 206 along the axis 205. The axis 205 may define a central longitudinal axis of the body 202. In some embodiments, the axis 205 is otherwise oriented, for example relative to the body 202 (e.g., extend from a first side surface to a second side surface, extend from a top surface to a bottom surface, etc.).

As shown in FIGS. 2-4, the body 202 further includes a base portion, shown as base 208. The base 208 may be positioned between the proximal end 204 and the distal end 206. For example, the base 208 may be positioned between the proximal end 204 and the distal end 206, toward the distal end 206 of the body 202 (e.g., as shown in at least FIG. 2). In some embodiments, the base 208 includes a tapered portion. For example, the base 208 may include a first portion that is substantially uniform thickness (e.g., extending from the proximal end 204 toward the distal end 206), and a second portion that tapers toward the distal end 206 (e.g., as shown in at least FIG. 2).

The body 202 further includes a first surface, shown as a top surface 210, and a second surface opposite the first surface, shown as a bottom surface 212 (e.g., as shown in at least FIG. 3). The top surface 210 and/or the bottom surface 212 may extend from the proximal end 204 to the distal end 206. The body 202 is shown to further include a third surface, shown as a first side surface 214, and a fourth surface opposite the third surface, shown as second side surface 216 (e.g., as shown in at least FIG. 3). The first side surface 214 may couple the top surface 210 and the bottom surface 212. The second side surface 216 may further couple the top surface 210 and the bottom surface 212. As shown in FIGS. 2-4, the first side surface 214 and the second side surface 216 may extend between the proximal end 204 and the distal end 206. In this regard, the top surface 210, the bottom surface 212, the first side surface 214 and/or the second side surface 216 may extend between the proximal end 204 and the distal end 206 and/or define the body 202.

The body 202 is also shown to include a working portion 220. The working portion 220 may be positioned at the distal end 206 of the body 202, extending from the base 208. For example, the working portion 220 may be positioned at the distal end 206 of the body 202 extending from the tapered portion of the base 208. The working portion 220 is shown to include a curved surface, for example a concave surface (e.g., opening toward the top surface 210, curved upward toward the top surface 210, etc.). In this regard, the working portion 220 may be or include a well, a depression, a hollow, a bowl, and/or another suitable configuration, for example to accommodate one or more non-planar surfaces of a bone. In other embodiments, the working portion 220 is another suitable configuration, shape, and/or design (e.g., a plate, a foot, a hook, a ledge, a flange, pronged plates, pronged feet, etc.). According to an exemplary embodiment, and as will be described herein, the working portion 220 is configured to be inserted into a joint space (e.g., to rest on a first bone, receive a second bone, etc.) and/or cause distraction of the joint (e.g., separation of the first and second bones of the joint).

The body 202 is further shown to include a handle portion 222. The handle portion 222 may be positioned at the proximal end 204 of the body 202, extending from the base 208. For example, the handle portion 222 may be positioned at the proximal end 204 of the body 202 extending from the substantially uniform portion of the base 208. The handle portion 222 may be curved (e.g., at an end of the proximal end 204), for example curved downward toward the bottom surface 212. In some embodiments, the handle portion 222 is of non-uniform thickness. For example, the handle portion 222 may decrease in thickness as the handle portion 222 extends from the base 208 to the proximal end 204. The handle portion 222 may also be of non-uniform width. For example, the handle portion 222 may increase in width as the handle portion 222 extends from the base 208 to the proximal end 204. According to an exemplary embodiment, and as will be described herein, the handle portion 222 may be configured to receive and/or transfer a force (e.g., torque, etc.) to the distal end 206 (e.g., the working portion 220, etc.), for example to cause distraction of the joint.

In other embodiments, the handle portion 222 is another suitable configuration, shape, and/or design (e.g., uniform, flat, curved upward, etc.). For example, in some embodiments the handle portion 222 includes a rotatable portion (e.g., between the base 208 and the proximal end 204, etc.). A rotatable portion of the handle portion 222 may allow for an alteration (e.g., modification, adjustment, variation, etc.) of the amount of force (e.g., torque, etc.) applied during a joint distraction. For example, when distracting a knee joint, the distraction force should be provided substantially parallel with a mechanical axis of the joint. However, a surgeon may not be able to achieve suitable access and/or grip on the distraction lever 200 such that all forces are applied in this direction. As a result, some force (e.g., torque, etc.) may instead be applied sideways on the joint, while also being applied in parallel with the mechanical axis. A rotatable portion of the handle portion 222 may allow for a reduction in the amount of force working laterally during joint distraction and/or may counteract some of the sideways force (e.g., torque, etc.) applied, for example by cooperating with twisting that may occur on the handle portion 222 when a force is applied at the proximal end 204.

Referring still to FIGS. 2-4, the body 202 is shown to include a measurement device, sensor, or gauge, shown as strain gauge 230. The strain gauge 230 may be positioned at the base 208 (e.g., above a compression aperture, etc.). For example, the strain gauge 230 may be positioned at the base 208, disposed within the top surface 210. A strain gauge 230 may additionally or alternatively be disposed within the bottom surface 212, in some embodiments. In some embodiments, the strain gauge 230 is disposed within a surface of the body 202 (e.g., the top surface 210 and/or the bottom surface 212, etc.) via one or more deposition processes (e.g., photodeposition, impregnation, etc.). According to an exemplary embodiment, the strain gauge 230 is configured to measure a force (e.g., torque, etc.) applied at one or more portions of the body 202 (e.g., the base 208, the working portion 220, the handle portion 222, etc.), for example when distracting the joint.

In an exemplary embodiment, the strain gauge 230 may be positioned (e.g., above a compression aperture, etc.) at any location where the distraction lever 200 (e.g., the body 202, etc.) exhibits suitable characteristics (e.g., material, one or more dimensions, etc.). For example, the strain gauge 230 may be positioned (e.g., above a compression aperture, etc.) at any location where a cross-sectional dimension of the distraction lever 200 (e.g., a cross-sectional height, etc.) is between 1.5 mm and 2.5 mm, and particularly 1.75 mm and 2.0 mm. In other embodiments, the strain gauge 230 may be positioned (e.g., above a compression aperture, etc.) at a location where a cross-sectional dimension (e.g., a cross-sectional height, width, length, area, etc.) of the distraction lever 200 is another suitable dimension (e.g., 1, 1.25, 1.75, 2.25, 2.75, etc. mm). In some embodiments, the strain gauge 230 may be positioned (e.g., above a compression aperture, etc.) at a location where the distraction lever 200 experiences a greatest force (e.g., stress, strain, torque, etc.) and/or where a largest characteristic (e.g., temperature, pressure, fluid flow, movement, etc.) is imparted or exhibited. For example, the strain gauge 230 may be positioned at a region between where the distraction lever 200 couples with one or more bones and where a force is applied to the distraction lever 200 (e.g., a base, a neck, etc.), a region between an aperture/opening and an edge or surface (e.g., where a force is applied, etc.), a region where growth material or movement occurs (e.g., in a cavity where healing or growth occurs, an area where components move or are manipulated, etc.), and/or another suitable area or region of the distraction lever 200.

As will be discussed herein, the flexibility in positioning of the strain gauge 230 (i.e., and the compression aperture, etc.) advantageously allows for obtaining measurements of characteristics (e.g., force, strain, temperature, pressure, etc.) at or around various locations of the distraction lever 200, while maintaining the integrity (e.g., structure, strength, stability, etc.) of the distraction lever 200, which allows for additional data collection and/or more precise measurements.

In some embodiments, the strain gauge 230 is coupled with a power source (e.g., a battery, an electrical outlet via a wired connection, etc.). As will be described herein, in an exemplary embodiment an electrical conductor of the strain gauge 230 is configured to deform. For example, as the distraction lever 200 deforms as a force is applied to distract the bones of the joint (e.g., the base 208 deforms, etc.), the electrical resistance of the electrical conductor of the strain gauge changes. Thus, from the measured electrical resistance of the strain gauge 230, the amount of applied stress to the distraction lever 200 (e.g., at the proximal end 204, the handle portion 222, etc.) can be determined (e.g., measured) and/or the distraction force applied at one or more bones of the joint can be computed (e.g., using known or measured input voltages, using measured output voltages, etc.).

For example, according to an exemplary embodiment the strain gauge 230 is configured to measure a force (e.g., torque, etc.) applied at a portion of the base 208 (e.g., a top wall, a top surface, a bottom wall, a bottom surface, etc.). In some embodiments, the strain gauge 230 is configured to measure a force (e.g., torque, strain, stress, etc.) on the top surface 210, for example caused by a torque applied between the distal end 206 and the proximal end 204. For example, where one or more external forces are applied at one or more points on the distraction lever 200 (e.g., a force applied by a user or operator at the proximal end 204, a force applied by the working portion 220 to a bone of the joint at the distal end 206, etc.), a surface of the distraction lever 200 (e.g., the top surface 210, the bottom surface 212, etc.) may deform (e.g., a bend, bow, bias, compress, etc., for example as a result of the external forces). The strain gauge 230 may be configured to detect (e.g., determine, measure, calculate, etc.) a value associated with the deformation at the surface (e.g., the top surface 210, etc.). For example, and as described herein, the deformation may result in one or more electrical properties of the strain gauge 230 to alter, for example enabling the strain gauge 230 to measure an amount of force (e.g., torque, strain, stress, etc.) at the portion of the base 208 (e.g., at the strain gauge 230, etc.).

In an exemplary embodiment, when a distance from the base 208 (e.g., the strain gauge 230) to the distal end 206 (e.g., a tip of the working portion 220, a bottom of the working portion 220, etc.) is known and/or selected, an applied distraction force can be determined (e.g., an applied force at the handle portion 222, etc.). In some embodiments, when a distance from the base 208 (e.g., the strain gauge 230) to the proximal end 204 (e.g., a tip of the handle portion 222, a tip of the proximal end 204, etc.) is known and/or selected, an applied distraction force can be determined (e.g., an applied force at the handle portion 222, etc.). Further, the applied distraction force can be used to calculate an applied distraction force at one or more bones of a joint (e.g., an applied force via the working portion 220, the distal end 206, etc.). In this regard, the selected and/or known arrangement of the base 208 (e.g., the strain gauge 230) relative to one or more components of the distraction lever 200 (e.g., a distance to the distal end 206, a distance to the proximal end 204, a distance to a base of the working portion 220, a distance to a base of the handle portion 222, etc., and/or any combination thereof) can be used to measure a force applied at one or more portions of the base 208 and/or to determine (e.g., calculate) an applied distraction force (e.g., to the proximal end 204, to the handle portion 222, at one or more bones of the joint, at the working portion 220, at the distal end 206, etc.).

In some embodiments, the body 202 includes a plurality of measurement devices, sensors, and/or gauges. For example, the body 202 may include the strain gauge 230 positioned at the base 208 (e.g., disposed within the top surface 210, etc.), as shown in at least FIG. 2. Further, the body 202 may include a strain gauge 232, for example positioned at the base 208 and/or disposed within the bottom surface 212 (e.g., as shown in at least FIG. 3). Similar to the strain gauge 230, the strain gauge 232 may be disposed within a surface of the body 202 (e.g., the bottom surface 212, etc.) via one or more deposition processes (e.g., photodeposition, impregnation, etc.). Further, the strain gauge 232 may be configured to measure a force (e.g., toque, etc.) applied at one or more portions of the body 202 (e.g., the base 208, the working portion 220, the handle portion 222, etc.), for example when distracting the joint.

As shown in FIGS. 2-3, the one or more measurement devices are communicably coupled with a communications device, shown as a wire 236 (e.g., as shown in at least FIG. 3). According to an exemplary embodiment, the strain gauge 230 and/or the strain gauge 232 is/are communicably coupled with the wire 236, for example to communicate one or more force measurements to a remote system and/or device (e.g., a robotic system, a computer system, a surgical system, etc.), as described herein. In other embodiments, the wire 236 is another suitable communications device and/or (e.g., a network, etc.), for example to facilitate wireless communication of one or more force measurements to a remote system and/or device.

As shown in FIG. 2, the wire 236 may be disposed within a channel, groove, or ridge of the body 202, shown as a channel 238. The channel 238 may extend from the base 208 to the proximal end 204 (e.g., along the axis 205, etc.), and may be configured to receive the wire 236. For example, the channel 238 may extend through the body 202 at the base 208 (e.g., from the bottom surface 212 to the top surface 210, etc.), for example to facilitate coupling the strain gauge 232 and the wire 236 (e.g., as shown in at least FIG. 3). Further, the channel 238 may extend into the top surface 210 and/or extend from the base 208 to the proximal end 204, for example to facilitate coupling the strain gauge 230 and the wire 236 (e.g., as shown in at least FIG. 2). Yet further, the channel 238 may extend through the body 202 at the proximal end 204 (e.g., from the top surface 210 to the bottom surface 212), for example to facilitate coupling with a remote system and/or device (e.g., a robotic device, a computing device, a surgical system, etc.).

In an exemplary embodiment, the channel 238 is configured to receive the wire 236, and the wire 236 may be potted within the channel 238. For example, the wire 236 may be received within the channel 238, and the wire 236 may be potted (e.g., encapsulated, etc.) with one or more solid or semi-solid compounds (e.g., polyurethane, silicone, etc.). In this regard, the wire 236 may be disposed (e.g., potted, encapsulated, etc.) within the channel 238, such that the wire 236 is shielded (e.g., protected, guarded, isolated, sheltered, etc.) from one or more external conditions (e.g., an external force on the top surface 210, cleaning or refurbishing compounds, corrosive agents, autoclaving, sterilization procedures, etc.). In other embodiments, the one or more measurement devices (e.g., the strain gauge 230, the strain gauge 232, etc.) and/or the communications device (e.g., the wire 236, etc.) is/are otherwise configured, arranged, and/or designed. For example, the strain gauges 230, 232 may be configured to communicate wirelessly (e.g., via Bluetooth, short-range wireless communications, RFID, etc.) and/or via another suitable communications technique, for example with one or more remote systems and/or devices described herein (e.g., a robotic device, a computing system, a surgical system, etc.).

It should be understood that while the one or more force measurement devices (e.g., the strain gauge 230, the strain gauge 232, etc.) are described herein as strain gauges disposed within a surface of the body 202, it is contemplated that the measurement devices may be another suitable component and/or device. For example, the force measurement device may include a piezoelectric pressure sensors, where a charge is generated when a piezoelectric crystal, or other suitable material, of the pressure sensor is stressed. The charge output, or the charge output converted to a voltage signal, for example, may be used to compute and/or indicate the distraction force being applied by the distraction lever. In other embodiments, the force measurement device may be an optical sensor (e.g., in a cantilever beam configuration, etc.). The optical sensor may include an array of optical fibers capable of providing computation of stress and strain by way of wavelength variations between the light source and a detector caused by modifications in the optical fiber body. Further, in other embodiments the force measurement device may include a magnetic contact switch, which may be used to indicate the presence of a load being applied and/or can be configured to indicate how much load is being applied.

Further, it should be understood that while the one or more force measurement devices (e.g., the strain gauge 230, the strain gauge 232, etc.) are described herein as strain gauges disposed within certain surfaces and/or at certain locations of the body 202, it is contemplated that the measurement devices may be otherwise oriented and/or positioned. For example, the strain gauge 230 may be disposed within the first side surface 214 (e.g., at the base 208) and/or the second strain gauge 232 may be disposed with the second side surface 216 (e.g., at the base 208). In other embodiments, the strain gauges 230, 232 may be positioned at another portion of the body 202 (e.g., the handle portion 222, the proximal end 204, etc.) and/or otherwise arranged (e.g., on adjacent surfaces, on the same surface, etc.). All such configurations are contemplated herein.

Referring still to FIGS. 2-4, the body 202 also includes a void, an aperture, an opening, or a gap, shown as compression aperture 240. The compression aperture 240 may be positioned between the proximal end 204 (e.g., the handle portion 222) and the distal end 206 (e.g., the working portion 220). For example, the compression aperture 240 may be positioned at the base 208 (e.g., proximate the strain gauge 230, the strain gauge 232, etc.). The compression aperture 240 may extend into and/or through the body 202, for example to create one or more openings in the body 202. Further, the compression aperture 240 and/or the one or more measurement devices (e.g., the strain gauge 230, the strain gauge 232) may be positioned adjacent one another. According to an exemplary embodiment, and as will be described herein, the compression aperture 240 is configured to displace and/or distribute one or more forces (e.g., torque) applied at one or more portions of the body 202 (e.g., the base 208, the working portion 220, the handle portion 222, etc.), for example when distracting the joint, for example to enable slight bending or other deformation of the top surface 210 and/or bottom surface 212 at the location of the strain gauge 230 and/or strain gauge 232.

According to an exemplary embodiment, the compression aperture 240 extends through the body 202. For example, the compression aperture 240 may extend through the body 202 (e.g., at the base 208) from the first side surface 214 to the second side surface 216. As shown in FIG. 2, the compression aperture 240 may have a substantially rectangular cross-sectional shape (e.g., a cross-sectional shape along the axis 205, parallel to the first side surface 214 and/or the second side surface 216, etc.). The cross-sectional shape of the compression aperture 240 may also include one or more circular shapes. For example, and as shown in FIG. 2, the cross-sectional shape of the compression aperture 240 may be rectangular with circular shapes at each corner. According to an exemplary embodiment, the shape, size, and/or configuration of the compression aperture 240 (e.g., the cross-sectional shape, etc.) is configured to selectively displace and/or distribute one or more forces (e.g., torque) at the base 208 (e.g., around the compression aperture 240).

According to an exemplary embodiment, the compression aperture 240 is defined (e.g., bound, confined, etc.) by one or more walls or surfaces. For example, the compression aperture 240 may be defined by a first wall or first surface, shown as a top wall 250, and a second wall or second surface (e.g., opposite the first wall or surface, etc.), shown as a bottom wall 252. The top wall 250 and/or the bottom wall 252 may include one or more cutouts or voids (e.g., circular cutouts, etc.), for example at opposing ends (e.g., a proximal end, a distal end, etc.). According to an exemplary embodiment, the top wall 250 traverses the compression aperture 240, for example forming a top portion of the base 208 that extends between the proximal end 204 (e.g., the handle portion 222) and the distal end 206 (e.g., the working portion 220). Similarly, the bottom wall 252 may traverse the compression aperture 240, for example forming a bottom portion of the base 208 that extends between the proximal end 204 (e.g., the handle portion 222) and the distal end 206 (e.g., the working portion 220).

As shown in FIG. 2, the one or more measurement devices are positioned at the base 208, for example adjacent the compression aperture 240. For example, the strain gauge 230 may be positioned at and/or disposed in the top wall 250 (e.g., as shown in at least FIG. 2). Similarly, the strain gauge 232 may be positioned at and/or disposed in the bottom wall 252 (e.g., as shown in at least FIG. 3). According to an exemplary embodiment, and as will be described herein, when distracting a joint a force is applied to the proximal end 204 (e.g., the handle portion 222), and the compression aperture 240 may cause the force to be displaced and/or distributed to one or more portions of the base 208, for example the top wall 250 and/or the bottom wall 252. The arrangement of the strain gauge 230 at the top wall 250 and/or the strain gauge 232 at the bottom wall 252, and the associated displacement and/or distribution of force during a distraction, allows the strain gauge 230 and/or the strain gauge 232 to measure one or more forces at the base 208.

According to an exemplary embodiment, the compression aperture 240 is further defined by a third wall or third surface, shown as a proximal wall 254, and a fourth wall or fourth surface (e.g., opposite the third wall or third surface), shown as distal wall 256. The proximal wall 254 and/or the distal wall 256 may couple the top wall 250 and the bottom wall 252. For example, the proximal wall 254 and/or the distal wall 256 may couple the top wall 250 and the bottom wall 252 (e.g., at the one or more cutouts or voids, etc.). According to an exemplary embodiment, the proximal wall 254 and/or the distal wall 256 are configured to support one or more portions of the base 208 (e.g., the top wall 250, the bottom wall 252, etc.), for example when a force is applied during a distraction of a joint.

It should be understood that in other embodiments, the compression aperture is another suitable configuration, shape, and/or design. For example, the compression aperture 240 may extend into and/or through another portion of the body 202 (e.g., a portion of the handle portion 222, etc.). The compression aperture 240 may extend into and/or through the body 202 in another orientation (e.g., from the top surface 210 to the bottom surface 212, etc.). In some embodiments, the cross-sectional shape of the compression aperture is another suitable shape and/or configuration (e.g., rectangular with circular shapes at opposing corners, rectangular with chamfered corners, rectangular, square, circular, oval, etc.). In other embodiments, the body 202 includes a plurality of compression apertures (e.g., 2, 3, 5, 10, etc.), which are suitably sized, arranged (e.g., extending into and/or through the body 202, etc.), oriented, and/or shaped. All configurations are contemplated herein.

Referring still to FIGS. 2-4, according to an exemplary embodiment the distraction lever 200 is any suitable size, shape, and/or is formed of one or more suitable materials. For example, the body 202 may be sized to be inserted into a joint space. In this regard, in some embodiments the body 202 is between 1.0 mm and 4.0 mm thick at the distal end 206 (e.g., the working portion 220, etc.). In some embodiments, the body 202 is approximately 2.05 mm thick at the distal end 206 (e.g., the working portion 220) and, in other embodiments, no more than approximately 3.50 mm thick. The width of the distal end 206 (e.g., the working portion 220, etc.) may be between 70.0 mm and 85.0 mm wide. In some embodiments, the width of the distal end 206 (e.g., the working portion 220) is approximately 81.0 mm wide to accommodate compartments of various sizes, and in a preferred embodiment, is approximately 74.0 mm wide.

In an exemplary embodiment, one or more components of the distraction lever 200 is/are formed of one or more biocompatible materials. For example, the distraction lever 200 may be formed of one or more suitable metals (e.g., stainless steel, carbon steel, titanium, aluminum, obsidian, etc.) and/or another suitable material (e.g., acetal copolymers, high-density polyethylene, nylon, polycarbonate, polyphenyl sulfone, polypropylene, etc.).

In an exemplary embodiment, one or more surfaces of the distraction lever 200 are treated with one or more materials. For example, a portion of a surface of the body 202 may be treated with a dialectic material. In an exemplary embodiment, the top surface 210 of the base 208 at a portion where the strain gauge 230 is positioned is treated with a suitable dielectric material. Similarly, the bottom surface 212 of the base 208 at a portion where the strain gauge 232 is positioned may be treated with a suitable dielectric material. For example, the top surface 210 and/or the bottom surface 212 of the base 208 may include one or more cavities, for example to receive the strain gauge 230 and/or the strain gauge 232, respectively. Prior to deposition of the gauges, the cavities may be treated with a suitable dielectric material (e.g., an oxide that bonds with the conductive material of the distraction lever 200 and/or the base 208, etc.). In some embodiments, the dielectric material is applied via a photodeposition process, and/or another suitable process (e.g., impregnation, etc.). Following treatment, the strain gauge 230 and/or the strain gauge 232 may be disposed within a surface of the base 208 (e.g., the top surface 210, the bottom surface 212, respectively, etc.), for example via a deposition processes (e.g., photodeposition, etc.). According to an exemplary embodiment, the dielectric material provides a non-conductive substrate to dispose the measurement device (e.g., the strain gauge 230, the strain gauge 232, etc.) upon, and/or provides a substrate that is unaffected by various external forces (e.g., high temperatures, moisture, etc., for example during autoclaving and/or sterilization processes).

In some embodiments, the distraction lever 200 includes one or more surface and/or exterior treatments and/or treatment materials. For example, the surgical tool may be treated with a material suitable for one or more cleaning processes (e.g., autoclaving, sterilization, etc.). In an exemplary embodiment, the distraction lever 200 is treated with silicone (e.g., having silicone disposed over the strain gauge 230, wire 236, etc.). For example, following deposition of the strain gauge 230 within the distraction lever 200 (e.g., a surface of the base 208, etc.), the distraction lever 200 may be treated with a potting compound (e.g., silicone, etc.), for example to seal and/or protect the strain gauge 230 (and/or the wire 236, etc.) within the distraction lever 200. In other embodiments, the distraction lever 200 is treated with another suitable material (e.g., a synthetic polymer, polyamide, polypropylene, polyoxymethylene, polytetrafluoroethylene, polyetherentherketone, polyphenylene sulfide, polysulfone, and polyether sulfone, etc.), for example to facilitate one or more suitable cleaning and/or disinfecting procedures (e.g., autoclaving, sterilization, etc.) by protecting the strain gauge 230, strain gauge 232, and/or other components of the distraction lever 200.

It should be understood that in some embodiments, the distraction lever 200 includes additional, fewer, and/or different working components. For example, in some embodiments the distraction lever 200 includes one or more indicators, for example to provide feedback (e.g., to a user, a robotic device, a surgical system, etc.) relating to an applied force (e.g., a force applied at a bone of a joint, etc.). The feedback may indicate the current value of the force being applied, it may be representative of a particular range of force values (e.g., provide feedback when a predetermined force value has been reached or exceeded, etc.), and/or may provide feedback to facilitate control of the distraction lever 200 (e.g., provide feedback to a user, a robotic device, a surgical system, etc., for example to prevent application of excessive or too much force during a distraction, etc.). In some embodiments, the indicator is configured to provide visual feedback (e.g., via a light, a LED, etc.), audio feedback (e.g., via a beep, a tone, a warning, etc.), haptic feedback (e.g., via vibration, movement, etc.), and/or any other suitable feedback.

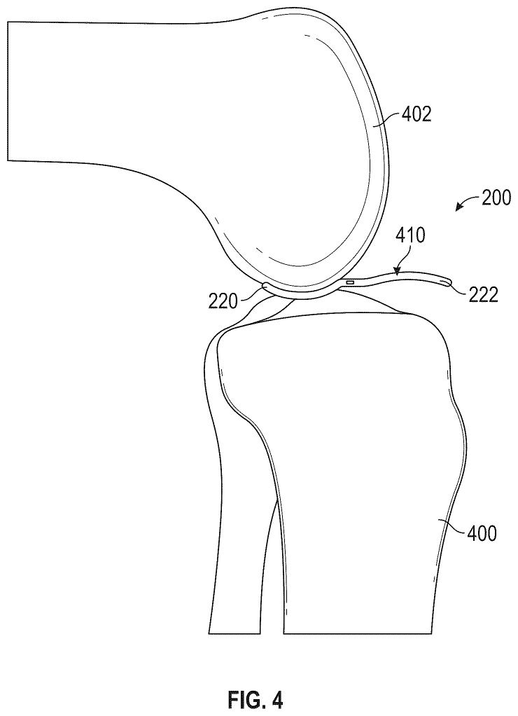

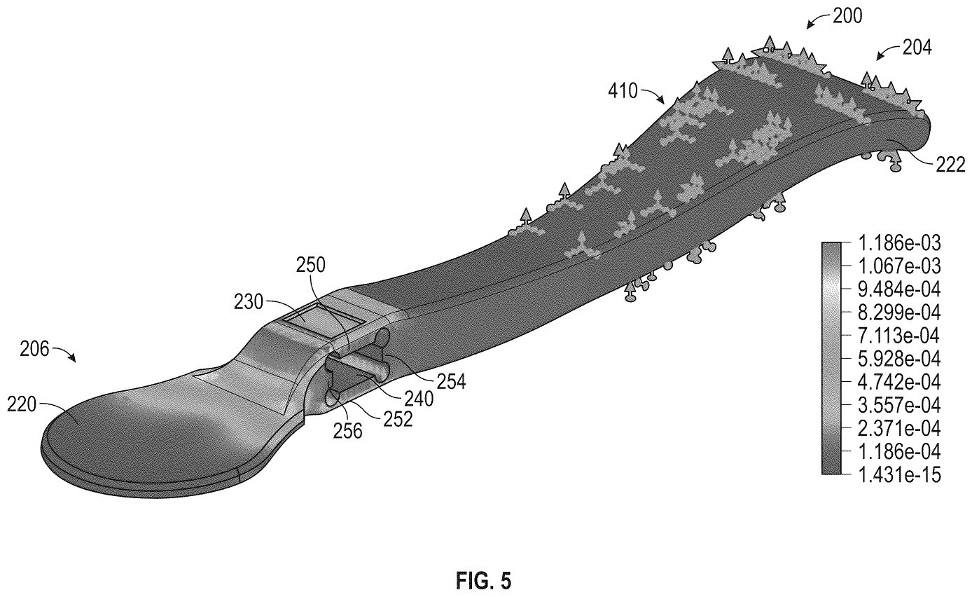

Referring now to FIGS. 4-5, an illustrative example of using the distraction lever 200 in a joint distraction of joint displacement procedure is shown, according to an exemplary embodiment. As described herein, it should be understood that while the embodiments of the distraction lever 200 are depicted and described as being used in a knee joint, the surgical tool may be used in any joint that is suitable for a joint distraction procedure.

As shown in FIG. 4, the distraction lever 200 may be manipulated such that the distraction lever 200 is positioned within a cavity of a joint. For example, the distraction lever 200 may be manipulated to position the working portion 220 within a knee cavity. For example, a first surface (e.g., a lower surface) of the working portion 220 may rest on a first bone (e.g., a longitudinal surface of a patient's tibia 400) and a second surface (e.g., a concaved or curved surface) of the working portion 220 may receive a second bone (e.g., a femoral condyle of a patient's femur 402).

With the working portion 220 within the joint cavity, a force 410 may be applied to the proximal end 204. For example, the force 410 may be applied (e.g., manually, via a robotic device, via a surgical system, etc.) to the handle portion 222 of the distraction lever 200. As shown in FIG. 5, the force 450 may be distributed across the handle portion 222 (e.g., the top surface 210 of the handle portion 222, etc.). In some embodiments, the force 410 is selected and/or determined, for example via control and/or guidance provided via a robotic device (e.g., the arm 112, etc.), a surgical system (e.g., the surgical system 100, etc.). In other embodiments, the force 410 is a manual force applied via a user or operator, which may be determined (e.g., calculated, etc.) via one or more measurements (e.g., via the strain gauge 230, etc.).

According to an exemplary embodiment, with the working portion 220 within the joint cavity, the force 410 applied to the proximal end 204 (e.g., the handle portion 222, etc.) can be transferred (e.g., distributed, provided, etc.) to the femur 402. For example, the force 410 may be transferred from the handle portion 222 to the working portion 220 (e.g., through the base 208) and to the femur 402. The force transferred to the femur 402 may cause distraction (e.g., separation, movement, etc.) of the femur 402 and the tibia 400. According to an exemplary embodiment, as the force 410 is applied to the handle portion 222, one or more force measurements may be determined by the one or more force measurement devices (e.g., the strain gauge 230, the strain gauge 232, etc.).

For example, and as described herein, when the force 410 is applied to the handle portion 222 and/or the working portion 220 applies a force to a bone of the joint (e.g., the femur 402, the tibia 400, etc.), the strain gauge 230 may be configured to detect (e.g., determine, measure, calculate, etc.) a value associated with a deformation at a surface (e.g., the top surface 210, etc.) of the distraction lever 200. For example, as the force 410 is applied to the handle portion 222 a surface of the distraction lever 200 may deform (e.g., bend, bow, bias, compress, etc.), for example as illustrated in FIG. 5. The deformation may alter one or more properties of the strain gauge 230 (e.g., electrical properties, etc.), for example enabling the strain gauge 230 to measure an amount of force (e.g., torque, strain, stress, etc.) at a portion of the base 208 during the distraction procedure. In this regard, in some embodiments, the force (or strain, torque, etc.) can be indirectly applied to the strain gauge 230 via the substrate on which it is formed (e.g., via the material of the base 208, of the top surface 210, etc.).

As also described herein, the force measurements may be used (e.g., via a robotic system, a surgical system, a computing system, etc.) to determine an applied distraction force. For example, the force measurements may be used to determine a distraction force applied at one or more bones of the joint (e.g., a force applied to the femur 402, a force applied to the tibia 400, etc.), a force applied to the handle portion 222 (e.g., determine a measure of a manual force applied, etc.), and/or another suitable applied force (e.g., a distributed or applied force at the base 208, the working portion 220, etc.).

As shown in FIG. 5, as the force 410 applied to the handle portion 222 is transferred (e.g., distributed, displaced, etc.) to the working portion 220, the force may be distributed and/or displaced at different portions of the distraction lever 200. For example, the compression aperture 240 may be configured to cause displacement and/or distribution of forces at one or more areas of the base 208. As shown in FIG. 5, application of the force 410 (e.g., with the working portion 220 positioned with the joint cavity, coupled between the femur 402 and the tibia 400, etc.) to the handle portion 222 results in an increased amount of force (e.g., torque, strain, stress, etc.) applied at the top wall 250 and/or the bottom wall 252, for example relative to the proximal wall 254 and/or the distal wall 256. As discussed herein, with the strain gauge 230 positioned at the top wall 250 and/or the strain gauge 232 positioned at the bottom wall 252, the strain gauges 230, 232 are configured to measure a force at the associated locations (e.g., the top wall 250, the bottom wall 252, etc.).

According to an exemplary embodiment, the arrangement of the compression aperture 240 (e.g., size, shape, orientation, etc.) is configured to selectively distribute and/or displace forces throughout the base 208, for example to facilitate more reliable and/or accurate measurements by the measurement devices (e.g., the strain gauge 230, the strain gauge 232, etc.). For example, by arranging the compression aperture 240 to cause an increased displacement and/or distribution of force to the top wall 250 and/or the bottom wall 252, the measurement devices (e.g., the strain gauge 230, the strain gauge 232, etc.) can be designed to measure larger forces (e.g., forces of more magnitude, etc.) and/or at smaller intervals. In this regard, the arrangement of the compression aperture 240 and/or the design of the strain gauges 230, 232 (e.g., designed and/or implemented via a photodeposition process, etc.) provides for more precise force measurements, which thereby allows for more accurate applied force determinations (e.g., calculations of forces applied at the joint, determinations of forces applied to the distraction lever 200, etc.).

As also shown in FIG. 5, in some embodiments the configuration and/or arrangement of the base 208 and/or the working portion 220 may be configured to cause displacement and/or distribution of forces at one or more areas. For example, a tapered region between the base 208 and the working portion 220 is shown to cause an increased amount of force (e.g., torque, strain, stress, etc.) applied at a portion of the working portion 220 proximate the base 208 (e.g., a neck or base region, the tapered region, etc.). According to an exemplary embodiment, the arrangement of the base 208 and/or the working portion 220 may facilitate a distribution and/or displacement of forces (e.g., a larger force at the region proximate the base 208, etc.), which results in a region of the working portion 220 that transfers force to bones of the joint (e.g., the femur 402, the tibia 400, etc.) having less strain (e.g., force, stress, etc.) relative to other regions of the working portion 220.

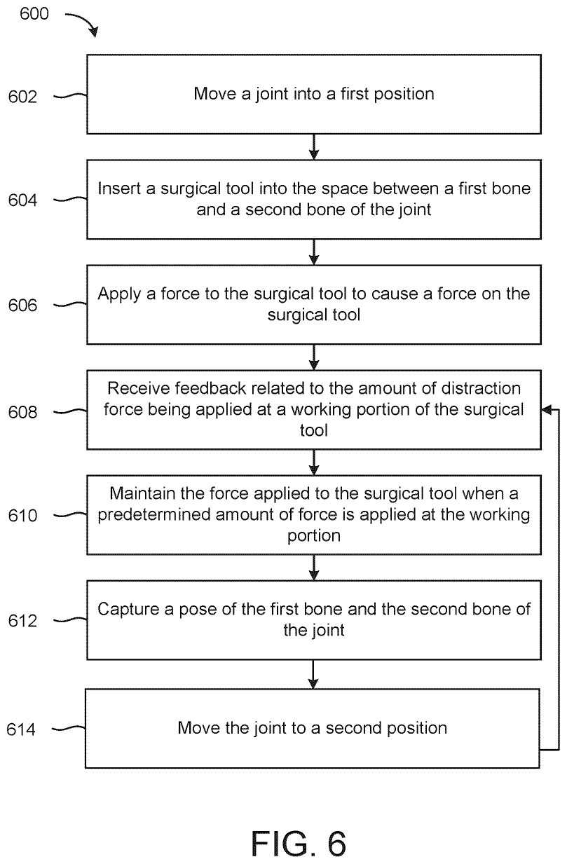

Referring now to FIG. 6, a method 600 for performing a joint distraction procedure is shown, according to an exemplary embodiment. In an exemplary embodiment, the method 600 is performed using the distraction lever 200 described herein. In some embodiments, the method 600 is performed using one or more components of the surgical system 100 described herein (e.g., the robotic system 102, the tracking system 118, etc.).

At step 602, a joint is moved to a first position. According to an exemplary embodiment, a joint, such as a knee joint including a tibia and a femur, is moved to a first flexion position. In some embodiments, the first position is a full flexion position, though any range of flexion may be used.

At step 604, a surgical tool is inserted into a space between a first bone and a second bone of the joint. According to an exemplary embodiment, the distraction lever 200 is inserted between the tibia 400 and the femur 402. For example, the working portion 220 may be inserted between the tibia 400 and the femur 402.

At step 606, a force is applied to the surgical tool to cause a force on the surgical tool. According to an exemplary embodiment, the force 410 is applied to the proximal end 204 of the distraction lever 200. For example, the force 410 may be applied to the handle portion 222 of the surgical tool, for example to cause force (e.g., torque, etc.) on the body 202 (e.g., the base 208, the working portion 220, etc.). In some embodiments, the force 410 is applied via a user (e.g., manually, etc.). In other embodiments, the force 410 is applied via one or more devices (e.g., a robotic system, the arm 112, a surgical system, etc.), as described herein.

At step 608, feedback is received relating to an amount of distraction force being applied at the working portion of the surgical tool. For example, a user and/or operator may receive feedback (e.g., a force measurement, etc.) relating to an amount of distraction force being applied at the working portion 220 (e.g., at the femur 402, at the tibia 400, etc.). In some embodiments, a user and/or operator may receive feedback (e.g., a force measurement, etc.) relating to an amount of force being applied at the proximal end 204 (e.g., the handle portion 222, etc.). According to an exemplary embodiment, the feedback is associated with measurements obtained by one or more measurement devices. For example, the feedback may be one or more force measurements obtained by the strain gauge 230 and/or the strain gauge 232.

In some embodiments, the feedback is received via one or more indicators and/or displays. For example, the feedback relating to the distraction force applied at the working portion 220 may be displayed via a display (e.g., the display device 106 of the surgical system 100, etc.). In other embodiments, the feedback is displayed via a user device (e.g., a computer, a mobile device, etc.). In other embodiments, the feedback is provided via one or more indicators provided on and/or along with the distraction lever 200.

At step 610, when a predetermined amount of force is applied at the working portion, the force applied to the surgical tool is maintained. For example, the user, having achieved a desired and/or having a desired awareness of the distraction force being applied at the working portion 220, may maintain the force 410 applied to the proximal end 204 (e.g., the handle portion 222, etc.). In some embodiments, the force 410 applied to the proximal end 204 (e.g., the handle portion 222, etc.) is maintained via the user (e.g., manually). In other embodiments, the force 410 applied to the proximal end 204 (e.g., the handle portion 222, etc.) is maintained via one or more devices (e.g., a robotic system, the arm 112, a surgical system, etc.), as described herein.

At step 612, a pose of the first bone and the second bone are captured. For example, with the predetermined force applied at the working portion 220 (e.g., the force 410 maintained at the handle portion 222, etc.) a pose of the femur 402 and/or a pose of the tibia 400 may be captured. According to an exemplary embodiment, capturing the pose of the first and/or second bones (e.g., the femur 402, the tibia 400, etc.) in the distracted joint assists with surgical planning, for example to ultimately attain the desired, properly aligned joint post-resection and post-prosthetic implantation. It is contemplated that to provide for capturing the pose of the joint the distraction lever 200 may be used in conjunction with anatomy navigation systems and methods (e.g., the tracking system 118), as described herein.

In some embodiments, the pose of the first bone and/or the second bone are used in conjunction with a surgical system (e.g., the surgical system 100). For example, the surgical system 100 may capture the pose of the femur 402 and/or the tibia 400, for example during a surgical procedure to detect movement and capture poses of the object(s). As described herein, using the pose data the surgical system 100 may also be configured to register (or map or associate) coordinates in one space to those in another to achieve spatial alignment or correspondence (e.g., using a coordinate transformation process as is well known).

In some embodiments, the surgical system 100 (e.g., the tracking system 118, etc.) is configured to track the anatomy and/or the distraction lever 200, for example while applying the distraction force. By tracking the pose and/or the movement of the distraction lever 200 and/or the anatomy (e.g., the femur 402, the tibia 400, etc.), the surgical system 100 can determine the directional components of the force being produced. As described herein, in addition to the forces acting along the mechanical axis, the distraction force may also act in a lateral direction or other direction off-axis from the mechanical axis. Tracking of the objects and determination of the directional components can allow for a determination of the amount of force that is off of the intended axis. This can help the surgeon, or the system automatically, to adjust the application of force for more efficient load transmission, and/or to reduce any injury or damage that may occur may applying distraction forces in directions that are off of the intended axis.

According to an exemplary embodiment, and as described herein, a virtual representation of the anatomy, such as the knee joint (e.g., the femur 402, the tibia 400, etc.), can be displayed on display device 106. The display device 106 may also display the relative position of the distraction lever 200 (e.g., relative to the femur 402, the tibia 400, etc.). Further, the display device 106 may also display one or more force measurements. For example, the display device 106 may displace one or more force measurements obtained from force measurement devices (e.g., strain gauge 230, strain gauge 232, etc.), a determined applied force (e.g., a distraction force applied at the working portion 220, etc.), a determined force received (e.g., a force applied at the handle portion 222, etc.), and/or any other suitable measurements.

In some embodiments, the pose data may also be used to complete a surgical planning procedure. For example, the surgical system 100 may capture and/or store the pose data of the first bone and/or the second bone. The captured pose data may be used to plan bone resection and prosthetic implant placement for proper joint balance and alignment. The surgical system 100 may define a surgical plan based on the captured pose(s) of the distracted joint. Further, the surgical system 100 may then implement the surgical plan, for example by tracking the distraction lever 200 relative to the patient's anatomy and/or may provide guidance throughout the procedure (e.g., haptic feedback, etc.).

At step 614, the joint is moved to a second position. According to an exemplary embodiment, the joint (e.g., the knee, the femur 402, the tibia 400, etc.) is moved to a second flexion position. In some embodiments, the joint may be moved to any number of positions (e.g., flexion positions, etc.). As shown in FIG. 6, steps 608-612 may be repeated. For example, the poses of the bones may be captured with the predetermined force (e.g., resection force, etc.) applied to the joint. In these poses, with the distraction force applied, the knee joint is in the desired post-resection final position. After collection of poses, bone resection, implant positioning, and implant characteristics are planned so as to maintain this relative alignment by making the femoral and tibial components contact (or be slightly gapped to allow for some laxity). Once the bone is resected at this desired plan and the trials and/or implants are secured to the bone, the leg will then be in the desired pre-resected posed positions.

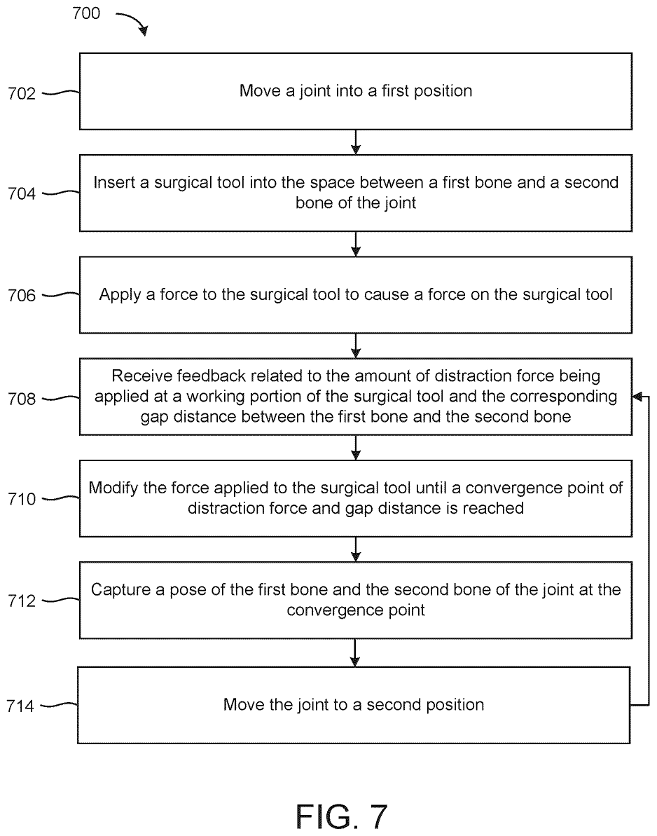

Referring now to FIG. 7, a method 700 for performing a gap balancing procedure is shown, according to an exemplary embodiment. In an exemplary embodiment, the method 700 is performed using the distraction lever 200 described herein. In some embodiments, the method 700 is performed using one or more components of the surgical system 100 described herein (e.g., the robotic system 102, the tracking system 118, etc.).

At step 702, a joint is moved to a first position. According to an exemplary embodiment, a joint, such as a knee joint including a tibia (e.g., the tibia 400, etc.) and a femur (e.g., the femur 402, etc.), is moved to a first flexion position. In some embodiments, the first position is a full flexion position, though any range of flexion may be used.

At step 704, a surgical tool is inserted into a space between a first bone and a second bone of the joint. According to an exemplary embodiment, the distraction lever 200 is inserted between the tibia 400 and the femur 402. For example, the working portion 220 may be inserted between the tibia 400 and the femur 402.

At step 706, a force is applied to the surgical tool to cause a force on the surgical tool. According to an exemplary embodiment, the force 410 is applied to the proximal end 204 of the distraction lever 200. For example, the force 410 may be applied to the handle portion 222 of the surgical tool, for example to cause force (e.g., torque, etc.) on the body 202 (e.g., the base 208, the working portion 220, etc.). In some embodiments, the force 410 is applied via a user (e.g., manually, etc.). In other embodiments, the force 410 is applied via one or more devices (e.g., a robotic system, the arm 112, a surgical system, etc.), as described herein.

At step 708, feedback is received relating to an amount of distraction force being applied at the working portion of the surgical tool. For example, a user and/or operator may receive feedback (e.g., a force measurement, etc.) relating to an amount of distraction force being applied at the working portion 220 (e.g., at the femur 402, at the tibia 400, etc.). In some embodiments, a user and/or operator may receive feedback (e.g., a force measurement, etc.) relating to an amount of force being applied at the proximal end 204 (e.g., the handle portion 222, etc.). According to an exemplary embodiment, the feedback is associated with measurements obtained by one or more measurement devices. For example, the feedback may be one or more force measurements obtained by the strain gauge 230 and/or the strain gauge 232.

In some embodiments, the feedback is received via one or more indicators and/or displays. For example, the feedback relating to the distraction force applied at the working portion 220 may be displayed via a display (e.g., the display device 106 of the surgical system 100, etc.). In other embodiments, the feedback is displayed via a user device (e.g., a computer, a mobile device, etc.). In other embodiments, the feedback is provided via one or more indicators provided on and/or along with the distraction lever 200.

According to an exemplary embodiment, feedback is received relating to an amount of distraction force applied at the working portion of the surgical tool and the corresponding gap distance between the first bone and the second bone. For example, a user and/or operator may receive feedback relating to a distance between the femur 402 and the tibia 400 (e.g., as a result of the distraction force, etc.). To measure the gap distance, the distraction lever 200 may be used in conjunction with anatomy navigation systems and methods (e.g., the tracking system 118), as described herein. For example, in some embodiments one or more trackers (e.g., a tibia tracker, a femur tracker, etc.) may be used to track a gap distance between the tibia 400 and the femur 402 (e.g., via the tracking system 118, etc.), during a distraction procedure.

At step 710, the force applied to the surgical tool is modified (e.g., as needed) until a convergence between the distraction force and the gap distance is reached. For example, the user, having an awareness of the distraction force being applied at the working portion 220 and/or a gap distance between the femur 402 and the tibia 400, may modify the force 410 applied to the proximal end 204 (e.g., the handle portion 222, etc.), for example until an optimal gap distance between the first bone (e.g., the tibia 400) and the second bone (e.g., the femur 402) is achieved. In some embodiments, the force 410 applied to the proximal end 204 (e.g., the handle portion 222, etc.) is modified via the user (e.g., manually). In other embodiments, the force 410 applied to the proximal end 204 (e.g., the handle portion 222, etc.) is modified via one or more devices (e.g., a robotic system, the arm 112, a surgical system, etc.), as described herein.

According to an exemplary embodiment, the optimal gap distance is correlated to the force 410 applied to the proximal end 204 (e.g., the handle portion 222, etc.). In other embodiments, an optimal gap distance between the first and second bones (e.g., the tibia 400, the femur 402, etc.) is determined based on feedback from one or more force measurement devices (e.g., the strain gauge 230, the strain gauge 232, etc.). As an example, the optimal gap distance can be determined by the juncture at which the force continues to rise, while the measured gap remains static. In some embodiments, the optimal gap distance corresponds to the maximum gap distance that can safely be achieved between the first and second bones (e.g., the tibia 400 and the femur 402, etc.). In other embodiments, the optimal gap distance corresponds to the greatest gap distance that is obtained without exceeding a certain force limit. The force limit may be predetermined, and/or may be determined based on feedback from the force measurement devices (e.g., the strain gauge 230, the strain gauge 232, etc.). As described herein, in some embodiments the gap distance is determined in combination with the tracking system 118.

At step 712, a pose of the first bone and the second bone are captured. For example, with the optimal gap distance achieved (e.g., a convergence between the distraction force and the gap distance, etc.), a pose of the femur 402 and/or a pose of the tibia 400 may be captured. According to an exemplary embodiment, capturing the pose of the first and/or second bones (e.g., the femur 402, the tibia 400, etc.) in the distracted joint assists with surgical planning, for example to ultimately attain the desired, properly aligned joint post-resection and post-prosthetic implantation. It is contemplated that to provide for capturing the pose of the joint the distraction lever 200 may be used in conjunction with anatomy navigation systems and methods (e.g., the tracking system 118), as described herein.

At step 714, the joint is moved to a second position. According to an exemplary embodiment, the joint (e.g., the knee, the femur 402, the tibia 400, etc.) is moved to a second flexion position. In some embodiments, the joint may be moved to any number of positions (e.g., flexion positions, etc.). As shown in FIG. 7, steps 708-212 may be repeated. For example, the user and/or operator may receive feedback related to the amount of distraction force being applied by the distraction lever 200 (e.g., at the working portion 220, etc.) and the gap distance between the first and second bone (e.g., the tibia 400, the femur 402, etc.) until the optimal gap distance is achieved, and/or the pose of the bones captured. After collection of poses, bone resection, implant positioning, and implant characteristics are planned so as to maintain this relative alignment by making the femoral and tibial components contact (or be slightly gapped to allow for some laxity). Once the bone is resected at this desired plan and the trials and/or implants are secured to the bone, the leg will then be in the desired pre-resected posed positions.

As described herein, the distraction lever 200 may be configured to be reusable. In this regard, the distraction lever 200 may be formed of one or more materials (e.g., as described herein), which is/are suitable for one or more cleaning techniques (e.g., autoclaving, sterilization techniques, etc.). As such, the distraction lever 200 may be reused in a series of one or more surgical processes and/or procedures, for example the processes described herein. For example, the distraction lever 200 may be used in a first joint distraction procedure (e.g., the method 600, etc.) for a first patient, cleaned (e.g., via autoclaving, a sterilization technique, etc.), and used again in a second joint distraction procedure (e.g., the method 600, etc.) for a second patient. Similarly, the distraction lever 200 may be used in a first gap balancing procedure (e.g., the method 700, etc.) for a first patient, cleaned (e.g., via autoclaving, a sterilization technique, etc.), and used again in a second gap balancing procedure (e.g., the method 700, etc.) for a second patient. In other embodiments, the distraction lever 200 is used in another suitable combination and/or sequence of procedures, for example as described herein.

While the examples above focus on a distraction lever, the present disclosure contemplates various types of surgical and medical instruments and devices having integrated strain gauges in accordance with the teachings herein, for example in which one or more strain gauges are disposed within (e.g., via photodeposition, impregnation, etc.) a substrate material (e.g., metal, plastic) forming a body of the surgical or medical instrument. Such instruments may include selective weakening, openings, etc. proximate the integrated strain gauge(s) to focus deformation at such locations thereby enabling force (torque, strain, etc.) measurements as described above for the distraction lever examples herein.

For example, the present disclosure contemplates instruments and devices having integrated strain gauges in accordance with the teachings herein, including, for example, packing, dressing, wraps, retractors, blades, implants, jacks (e.g., expandable jack implants, etc.), anchors, clips, fuses, wedges, plugs, fixation systems, cannulas, guidewires, encasements, reamers, cutters, burs, bits, drills, saw blades, allograft implants, synthetic implants, distractors, extenders, lengtheners, balloons, needles, screws, plates, sheathes, catheters, sutures, zips, support surfaces (e.g., mats, chairs, beds, stretchers, etc.), and/or other suitable instruments or devices. All such variations are within the scope of the present disclosure.

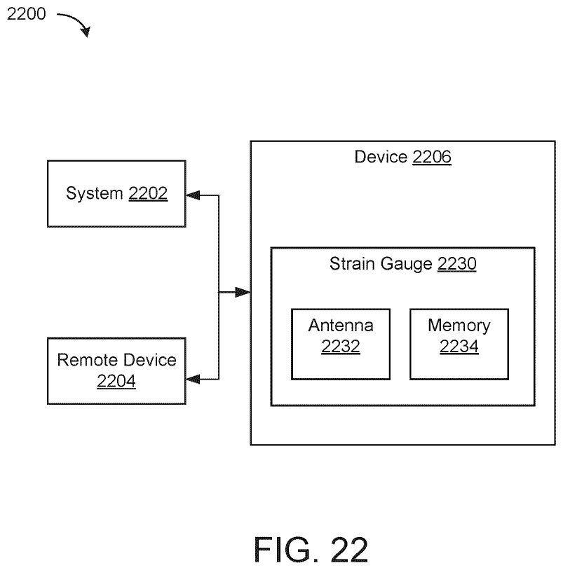

Referring generally to FIGS. 8-22, one or more devices, systems, and/or implants are shown, according to various embodiments. In an example embodiment, the devices of FIGS. 8-21 and/or the system of FIG. 22 may be implemented as one or more components of the surgical system 100 of FIG. 1. For example, one or more devices of FIGS. 8-21 may be configured to removably couple the robotic system 102 (e.g., the arm 112, etc.), for example such that the surgical system 100 (e.g., the robotic system 102, the arm 112, etc.) is used to guide and/or implant (e.g., insert, couple, etc.) the one or more devices of FIGS. 8-21 within a patient (e.g., to hold an implant in a planned position to facilitate coupling of the implant to one or more bones in the planned position). Further, the one or more devices of FIGS. 8-21 may be used in combination with the distraction lever 200, for example to implement one or more of the processes and/or techniques discussed herein.

Still referring generally to FIGS. 8-21, the devices of FIGS. 8-21 may include one or more strain gauges and one or more compression apertures. The strain gauges may be the same as or similar to the strain gauge 230, 232, and/or the compression apertures may be the same as or similar to the compression apertures 240, as discussed herein. For example, the devices of FIGS. 8-21 may include one or more integrated strain gauges, which are disposed within (e.g., via photodeposition, impregnation, etc.) a substrate or a material (e.g., metal, plastic, etc.) that forms the devices, and/or which are positioned relative to one or more compression apertures (e.g., adjacent to, above, below, etc.) in the device, for example in accordance with the teachings discussed with reference to FIGS. 1-7.

As described herein, the strain gauges and/or the compression apertures of the devices of FIGS. 8-21 may facilitate measuring (e.g., determining, collecting, calculating, computing, etc.) a value of a parameter (e.g., stress, strain, torque, pressure, temperature, movement, etc.) at and/or surrounding the devices. For example, the strain gauges may be used to determine (e.g., measure, collect, etc.) a value of a parameter (e.g., a stress, a strain, a temperature, a movement, etc.) at (or of) one or more components of the device itself (e.g., a surface, an end, a body, a base, etc. of the device, etc.). Further, the strain gauges may be used to determine a value of a parameter of one or more features coupled to, adjacent to, and/or surrounding the device (e.g., a bone or bone structure coupled with the device, a tissue or graft around the device, etc.), as described elsewhere herein. Yet further, and as described with reference to FIGS. 2-4, the ability to freely/selectively position the strain gauges and/or compression apertures relative to features of the devices of FIGS. 8-21 (e.g., at any location where the device exhibits a suitable cross-sectional dimension, or height, etc.), allows for a greater number and/or more precise measurement of characteristics (e.g., force, strain, pressure, temperature, movement, etc.) at or around different locations of the devices, while maintaining the integrity (e.g., structure, strength, stability, etc.) of the devices. All such embodiments are contemplated herein.

In an exemplary embodiment, one or more components of the devices of FIGS. 8-21 may be configured to communicate with a component of the surgical system 100 of FIG. 1. For example, the devices of FIGS. 8-21 (e.g., the strain gauges, etc.) may be configured to communicate data (e.g., date that is collected, measured, gathered, stored, determined, etc.) to a system and/or device (e.g., a robotic system, a computer system, a surgical system, etc.), as described herein. The data may be communicated wirelessly (e.g., via a short-range communication, inductive resonance, passive resonance, etc.), via a wired connection, and/or using any other suitable communications protocol, as also discussed herein. In some embodiments, the data is communicated continuously (e.g., in real-time, or near real-time, etc.), at periodic or predetermined intervals (e.g., hourly, daily, weekly, etc.), and/or at another suitable time (e.g., in response to an instruction, in response to a call or batch collection from a remote device, in response to induction of a current in circuitry of the device, etc.).

According to an exemplary embodiment, the devices of FIGS. 8-21 may be configured to communicate information (e.g., data that is collected, measured, determined, stored, etc.), for example to facilitate monitoring, analysis, and/or evaluation of one or more characteristics associated with the devices of FIGS. 8-21. More specifically, in an example embodiment the devices of FIGS. 8-21 may be implanted into an individual, collect information associated with characteristics of the device and/or surrounding bone/tissue/anatomical features (e.g., via one or more strain gauges to collect information associated with stress, strain, temperature, pressure, movement, etc.), and/or communicate that information (e.g., to a computing system, etc.), for example for monitoring, analysis, and/or evaluation.

As described herein, it is contemplated that the information from the devices of FIGS. 8-21 may be used for, for example, continuous, real-time, and/or near real-time monitoring of characteristics of the device and/or associated anatomical features. For example, the information may be used to monitor real-time characteristics (e.g., performance, condition, degradation, etc.) of the devices of FIGS. 8-21. In this regard, the information may be used to monitor whether the device was implanted correctly, how the device is performing after implantation and/or during activity, a stability or degradation status of the device, and/or other characteristics of the device. In other embodiments, the information may also be used to monitor characteristics (e.g., performance, condition, degradation, etc.) of surrounding anatomical features (e.g., bones, tissues, joints, fluids, etc.), for example to evaluate healing, repair, and/or biological reaction conditions associated with implanting the device. All such information may be collected and/or analyzed while the devices remain within the patient (e.g., at the implant site, at its implant location, at its natural position or location, etc.). In this regard, the information collected and/or communicated from the devices of FIGS. 8-21 may advantageously be used to proactively assess and/or address conditions associated with the devices and/or surrounding anatomical features (e.g., potential complications, degradation, endpoints indicating progression through stages of healing, etc.), without additional procedures that need/utilize access to the implant site.

It should be understood that this information collection, communication, and/or assessment may also advantageously offer earlier detection of potential complications (e.g., implant loosening, bone fracture, healing issues, etc.) and/or optimize patient rehabilitation schedules (e.g., by adjusting activity levels, rehabilitation schedules, implant configurations or positions, etc.) compared to traditional evaluation and/or assessment techniques, which may involve periodic assessment and/or non-invasive forms of data collection. Further, the information collection, communication, and/or assessment afforded herein advantageously allows for evaluation of additional data and/or information associated with the interactions between anatomical features and/or implants, which may be utilized to improve clinical outcomes, surgical planning, and/or implant development and design.

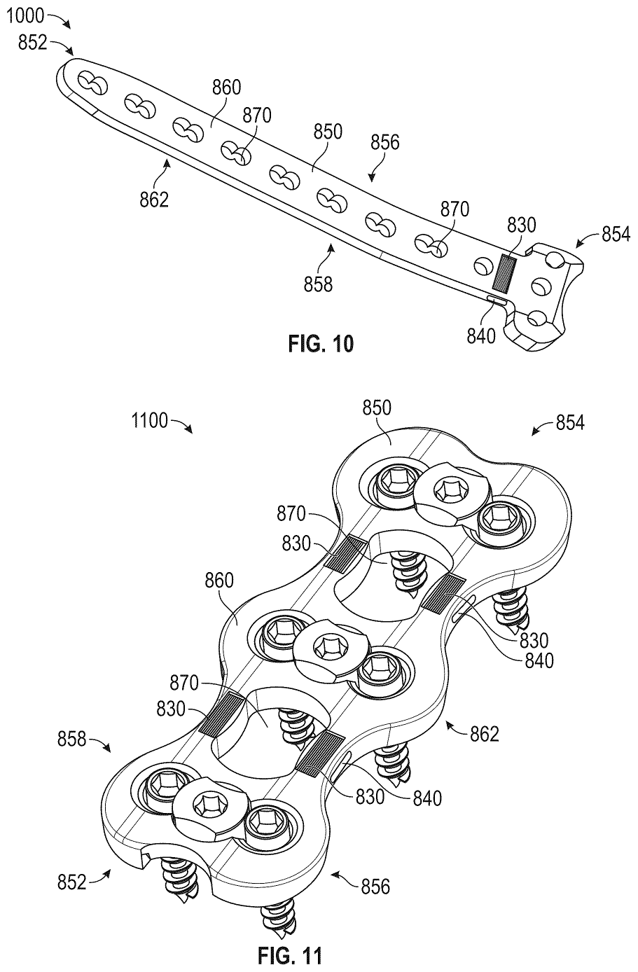

Referring now to FIGS. 8-11, a plurality of implants, implantable devices, and/or prosthetic devices are shown, according to various embodiments. In an exemplary embodiment, the devices of FIGS. 8-11 are plates. For example, the plate may be a broad plate or a simple broad plate, shown as plate 800 (e.g., as shown in FIG. 8), a first tibia plate, a distal tibia plate, or a medial-distal tibia plate, shown as plate 900 (e.g., as shown in FIG. 9), a second tibia plate, an anterior tibia plate, or an anterior tibia fixation plate, shown as plate 1000 (e.g., as shown in FIG. 10), and/or a cervical plate, or an anterior cervical plate, shown as plate 1100 (e.g., as shown in FIG. 11). In an exemplary embodiment, the plates of FIGS. 8-11 include one or more strain gauges, shown as strain gauges 830, and one or more compression apertures, shown as compression apertures 840. As will be discussed herein, the strain gauges 830 may be the same as or similar to the strain gauges 230, 232, and the compression apertures 840 may be the same as or similar to the compression apertures 240, as described herein with reference to FIGS. 1-7. In this regard, the compression apertures 840 and/or the strain gauges 830 may be specifically positioned relative to one another, and/or relative to other components of the devices of FIG. 8-11, as described herein.

In an exemplary embodiment, each of the plates 800, 900, 1000, and/or 1100 of FIGS. 8-11 include one or more features and/or components, which may be similar and/or the same. For example, each of the plates 800, 900, 1000, and 11000 are shown to include a body 850, which includes and/or is defined by a first end 852 and a second end 854 opposite the first end 852, a first side 856 and a second side 858 opposite the first side 856, a top surface 860 and a bottom surface 862 opposite the top surface 860. In some embodiments, one or more of the ends may be shaped as a nose, a tip, a foot, a ledge, a head, a T-shape configuration, and/or another suitable component or configuration. For example, the first end 852 may be shaped as a tip or nose (e.g., as shown in the plate 1000 of FIG. 10), and/or the second end 854 may be shaped as a foot (e.g., as shown in the plate 900 of FIG. 9) and/or a T-shaped configuration (e.g., as shown in plate 1000 of FIG. 10).

As shown in FIGS. 8-11, the body 850 may define one or more axis (e.g., a longitudinal axis extending between the first end 852 and the second end 854, a lateral axis extending between the first side 856 and the second side 858, etc.). Further, the body 850 is shown to include and/or define one or more apertures, shown as apertures 870. The apertures 870 may be configured to receive one or more anchoring and/or stabilization members (e.g., pins, screws, bolts, anchors, sutures, stitching, etc.), as shown in at least plate 1100 of FIG. 11, for example to secure the body 850 (e.g., the plates 800, 900, 1000, etc.) to one or more bones, bone surfaces, and/or anatomical features (e.g., tissue, tendons, cartilage, etc.). In some embodiments, the apertures 870 are also configured to receive one or more fluids and/or growth material (e.g., bone growth material, a graft, etc.), for example to facilitate healing and/or repair (e.g., of surrounding bone, tissue, and/or other anatomical features, etc.).

As also shown in FIGS. 8-11, the apertures 870 may be arranged or configured throughout the body 850. For example, the apertures 870 may be aligned along an axis of the body 850 (e.g., along/relative to a longitudinal axis of the plate, as illustrated in the plate 900 of FIG. 9, the plate 1000 of FIG. 10, the plate 1100 of FIG. 11, etc.), and/or the apertures 870 may be offset relative to one another along the body 850 (e.g., offset along a longitudinal axis of the plate, as illustrated in the plate 800 of FIG. 8). In some embodiments, the apertures are arranged in one or more patterns, which may be the same and/or different at different portions of the body 850 (e.g., in a more spread pattern at an end, as shown in the plate 900 of FIG. 9; in a pattern directed in a different orientation at an end, as shown in the plate 1000 of FIG. 1000, etc.).

As discussed herein, the body 850 of the plates of FIGS. 8-11 includes one or more strain gauges 830, which may be the same as or similar to the strain gauge 230, 232 of FIGS. 1-7, and/or one or more compression apertures 840, which may be the same as or similar to the compression apertures 240 of FIGS. 1-7, as described herein. For example, the body 850 may include one strain gauge 830 positioned at a surface of the body 850 (e.g., a strain gauge positioned at a top surface, as shown in plate 900 of FIG. 9 and plate 1000 of FIG. 10). In some embodiments, the body 850 includes a strain gauge 830 with a compression aperture 840 positioned adjacent the strain gauge 830 (e.g., as shown in plate 900 of FIG. 9 and plate 1000 of FIG. 10, etc.). In some embodiments, the compression aperture 840 extends through the body 850 (e.g., as shown in plate 800 of FIG. 8, etc.) and/or extends into a portion of the body 850 (e.g., as shown in plate 800 of FIG. 8, etc.), thereby providing reduced rigidity at the position of the strain gauge 830 or otherwise increase the deformity of the plate at the strain gauge 830 so as to improve the ability of the strain gauge 830 to measure strain on the plate. In other embodiments, the body 850 includes a strain gauge 830 without a compression aperture 840 (e.g., as shown in plate 1100 of FIG. 11, etc.), for example due to the positioning and/or characteristics of the body 850 where the strain gauge 830 is positioned.

In some embodiments, the body 850 includes a plurality of strain gauges 830 positioned at one or more surfaces of the body 850, and/or a plurality of compression apertures 840 (e.g., a plurality of strain gauges positioned at a top surface, as shown in plate 800 of FIG. 8 and plate 1100 of FIG. 11, etc.). The strain gauges 830 and/or compression apertures 840 may also be positioned relative to one or more components and/or portions the plates of FIGS. 8-11 (e.g., relative to the body 850). For example, the strain gauges 830 may be positioned at one or more edges or sides of the plate (e.g., a first side and/or a second side of the plate, as shown in the plate 800 of FIG. 8 and the plate 1100 of FIG. 11, etc.), which may or may not be positioned relative to one or more apertures 870 (e.g., between a side and an aperture of the plate, as shown in the plate 1100 of FIG. 11, etc.). As discussed herein, the strain gauges 830 may or may not be positioned adjacent one or more compression apertures 840 (e.g., as shown in the plate 1100 of FIG. 11, etc.). Further, the strain gauges 830 and/or compression apertures 840 may also be positioned between one or more areas or regions of the plate (e.g., at a neck region between a foot and a body region, as shown in the plate 900 of FIG. 9; at a neck region between T-head and T-body regions, as shown in plate 1000 of FIG. 10). In some embodiments, the strain gauges 830 and/or compression apertures 840 are positioned relative to and/or between one or more apertures 870 of the plate (e.g., as shown in the plates of FIGS. 8-10).

It should be understood that it is contemplated that the strain gauges 830 and/or compression apertures 840 may be or include any other suitable number of strain gauges and/or compression apertures (e.g., 3, 4, 5, 8, 10, 15, etc.), which may be otherwise positioned, oriented, and/or configured relative to one or more components of a plate (e.g., the plates of FIGS. 8-11). All such embodiments are contemplated herein.

According to an exemplary embodiment, and as described herein, the strain gauges 830 and compression apertures 840 of FIGS. 8-11 are configured to measure (e.g., determine, collect, calculate, compute, etc.) a value of one or more parameters (e.g., stress, strain, torque, pressure, temperature, movement, etc.) at and/or surrounding the plates of FIGS. 8-11. For example, the strain gauges 830 may be configured to determine (e.g., measure, collect, etc.) a value of one or more parameters (e.g., a stress, a strain, a temperature, a movement, etc.) of the plate itself. Further, the strain gauges 830 may be configured to determine a value of one or more parameters (e.g., pressure, temperature, etc.) associated with an anatomical feature surrounding the plate (e.g., a strain imparted on/by a bone coupled with the plate, a temperature of a surrounding tissue, a movement of an adjacent graft, etc.). As described herein, this information may be communicated (e.g., to a computing system, etc.), for example to facilitate monitoring, analysis, and/or evaluation of one or more characteristics associated with the devices and/or surrounding anatomical features.

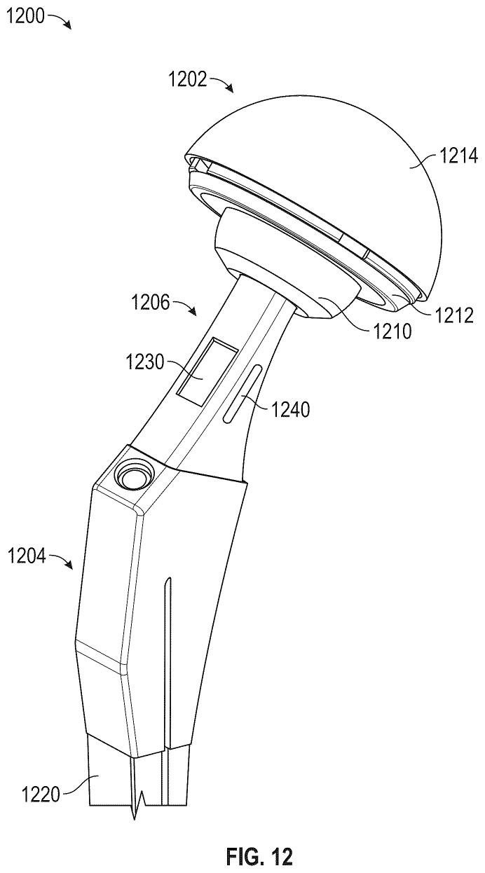

Referring now to FIG. 12, an implant, implantable device, and/or prosthetic device, shown as implant 1200, is shown, according to an exemplary embodiment. In an exemplary embodiment, the implant 1200 is a prosthesis or a prosthetic device, for example for a hip replacement surgery. It should be understood that while implant 1200 is described herein as being a hip prosthesis, for example for use in a hip procedure, it is contemplated that in other embodiments the implant 1200 is another suitable implant or device, for example for use in a variety of other surgical procedures described herein (e.g., shoulder, knee, hand, wrist, foot, ankle, vertebral, cranial, neurological, etc. implants or devices).

As shown, the implant 1200 includes a first area or region, shown as a head region 1202, and a second area or region, shown as a stem region 1204, with a connection area, shown as neck 1206, therebetween. The head region 1202 may include one or more components, which may facilitate coupling the implant 1200 with one or more bones and/or movement of the implant 1200. For example, the head region 1202 is shown to include a head 1210, a liner 1212, and a shell 1214. The head 1210 is shown to be coupled with the neck 1206, and may be received by the liner 1212. The liner 1212 may be received by the shell 1214, which may be configured to couple (e.g., be received by, etc.) one or more bones, tissues, and/or anatomical features (e.g., components of the acetabulum, etc.). In an exemplary embodiment, the engagement between the neck 1206, the head 1210, the liner 1212, and/or the shell 1214 is configured to facilitate, and allow movement that is similar to movement at the hip joint.

As shown, the neck 1206 extends between the head region 1202 (e.g., the head 1210) and the stem region 1204. The stem region 1204 is shown to include a stem 1220, which may be configured to couple (e.g., be received by, etc.) one or more bones, tissues, and/or anatomical features (e.g., a bore or channel within the femur, etc.). It is contemplated that in other embodiments, one or more components of the implant 1200 are otherwise arranged and/or configured, for example to facilitate use in a variety of other surgical procedures described herein (e.g., shoulder, knee, hand, wrist, foot, ankle, vertebral, cranial, neurological, etc.).

As shown, the implant 1200 includes one or more strain gauges, shown as strain gauge 1230, and one or more compression apertures, shown as compression aperture 1240. The strain gauge 1230 may be the same as or similar to the strain gauges 230, 232, and the compression aperture 1240 may be the same as or similar to the compression aperture 240, as described herein with reference to FIGS. 1-7. In this regard, the compression aperture 1240 and/or the strain gauge 1230 may be specifically positioned relative to one another, and/or relative to other components of the implant 1200 of FIG. 12, as described herein.

In an exemplary embodiment, the implant 1200 includes one strain gauge 1230 positioned at a surface of the neck 1206 (e.g., a strain gauge positioned at a top surface, as shown in FIG. 12). As shown, the neck 1206 also includes the compression aperture 1240 positioned adjacent the strain gauge 1230.

In some embodiments, the implant 1200 includes a plurality of strain gauges 1230 and/or compression apertures 1240. For example, the implant 1200 may include a plurality of strain gauges 1230 and/or compression apertures 1240 positioned throughout the neck 1206 (e.g., adjacent the head 1210, adjacent the stem region 1204, on a bottom surface, on one or more side surfaces, etc.). In some embodiments, the implant 1200 includes one or more strain gauges 1230 and/or compression apertures 1240 positioned throughout the head region 1202 (e.g., at the head 1210, the liner 1212, etc.) and/or throughout the stem region 1204 (e.g., at a proximal end of the stem 1220, at a distal end of the stem 1220, etc.). It should be understood that it is contemplated that the strain gauges 1230 and/or compression apertures 1240 may be or include any other suitable number of strain gauges and/or compression apertures (e.g., 3, 4, 5, 8, 10, 15, etc.), which may be otherwise positioned, oriented, and/or configured relative to one or more components of the implant 1200. All such embodiments are contemplated herein.

According to an exemplary embodiment, and as described herein, the strain gauge 1230 and compression aperture 1240 of the implant 1200 of FIG. 12 are configured to measure (e.g., determine, collect, calculate, compute, etc.) a value of one or more parameters (e.g., stress, strain, torque, pressure, temperature, movement, etc.) at and/or surrounding the implant 1200. For example, the strain gauge 1230 may be configured to determine (e.g., measure, collect, etc.) a value of one or more parameters (e.g., a stress, a strain, a temperature, a movement, etc.) of the implant 1200 itself. Further, the strain gauge 1230 may be configured to determine a value of one or more parameters (e.g., pressure, temperature, etc.) associated with an anatomical feature surrounding the implant 1200 (e.g., a strain imparted on/by a bone coupled with the implant, a temperature of a surrounding tissue, a movement of an adjacent graft, etc.). As described herein, this information may be communicated (e.g., to a computing system, etc.), for example to facilitate monitoring, analysis, and/or evaluation of one or more characteristics associated with the implant and/or surrounding anatomical features.

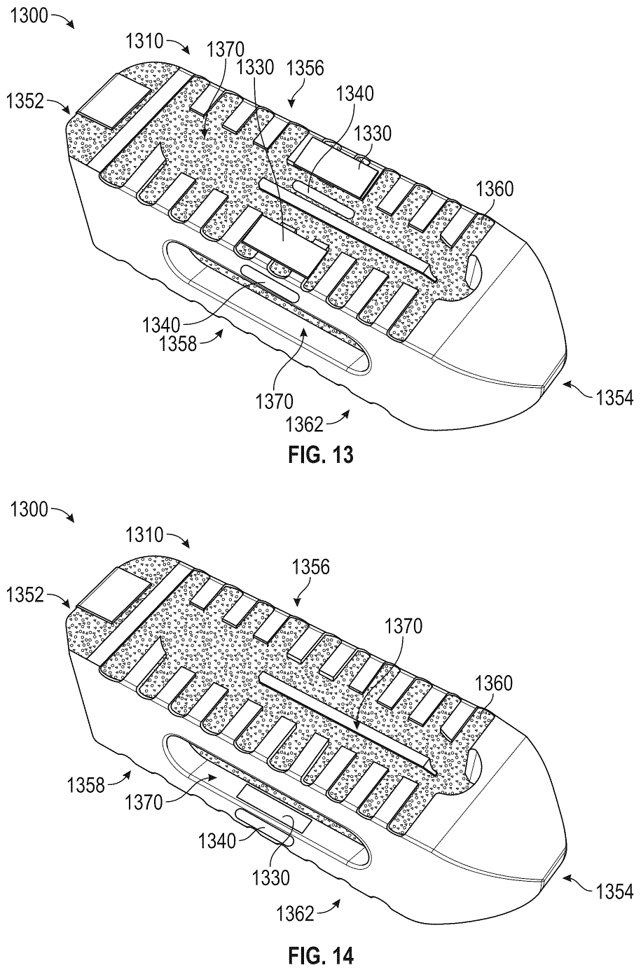

Referring now to FIGS. 13-16, a plurality of implants, implantable devices, and/or prosthetic devices are shown, according to various embodiments. In an exemplary embodiment, the devices of FIGS. 13-16 are cages (e.g., intervertebral cages, etc.). For example, the cage may be an intervertebral cage, an interbody cage, or an interbody fusion cage, shown as cage 1300, or the cage may be an expandable interbody cage, or an expandable implant, shown as cage 1500. In an exemplary embodiment, the cages of FIGS. 13-16 include one or more strain gauges, shown as strain gauges 1330, and one or more compression apertures, shown as compression apertures 1340. The strain gauges 1330 may be the same as or similar to the strain gauges 230, 232, and the compression apertures 1340 may be the same as or similar to the compression apertures 240, as described herein with reference to FIGS. 1-7. In this regard, the compression apertures 1340 and/or the strain gauges 1330 may be positioned relative to one another, and/or relative to other components of the devices of FIG. 13-16, as described herein.

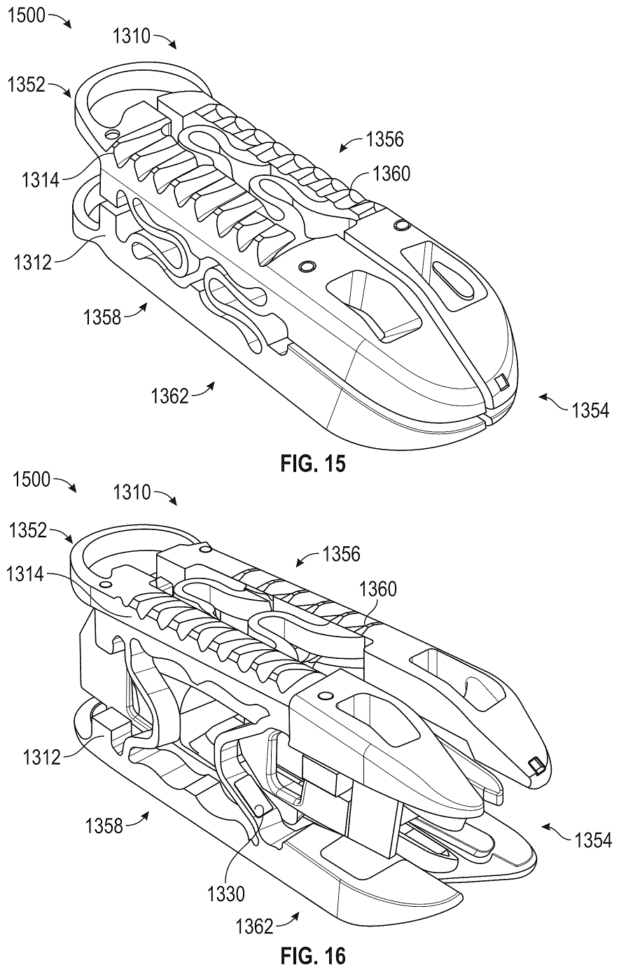

In an exemplary embodiment, each of the cages 1300, 1500 of FIGS. 13-16 include one or more features and/or components, which may be similar and/or the same. For example, each of the cages 1300, 1500 are shown to include a base 1310. In some embodiments, the base 1310 is a single base component (e.g., as shown in FIGS. 13-14). In other embodiments, the base 1310 includes a first base component (e.g., shown as a first base member 1312 in FIGS. 15-16) and a second base component (e.g., shown as a second base member 1314 in FIGS. 15-16), which may be movable relative to one another, for example between a first, collapsed, configuration (e.g., as shown in FIG. 15) and a second, expanded, configuration (e.g., as shown in FIG. 16). In an exemplary embodiment, each of the cages 1300, 1500 are configured to engage adjacent surfaces (e.g., adjacent surfaces of adjacent bone, etc.), for example to position one or more bones relative to one another (e.g., adjacent bones, adjacent vertebrae, etc.). In an exemplary embodiment, the cage 1500 includes additional components (e.g., expansion members, control members, driving members, etc.), for example to facilitate movement of the cage 1500 between the first, collapsed, configuration (e.g., as shown in FIG. 15) and the second, expanded, configuration (e.g., as shown in FIG. 16).

As shown in FIGS. 13-16, the base 1310 includes and/or is defined by a first end 1352 and a second end 1354 opposite the first end 1352, a first side 1356 and a second side 1358 opposite the first side 1356, and a top surface 1360 and a bottom surface 1362 opposite the top surface 1360. In some embodiments, one or more of the ends may be shaped as a nose, a tip, a foot, a rear, a driver end, and/or another suitable component or configuration. For example, the second end 1354 may be shaped as a tip or nose a foot (e.g., as shown in the cage 1300 of FIGS. 13-14 and the cage 1500 of FIGS. 15-16, etc.).

As shown in FIGS. 13-16, the base 1310 (and/or components thereof) may define one or more axis (e.g., a longitudinal axis extending between the first end 1352 and the second end 1354, a lateral axis extending between the first side 1356 and the second side 1358, etc.). Further, the base 1310 is shown to include and/or define one or more apertures, openings, spaces, voids, or channels, shown as cavities 1370. The cavities 1370 may extend into the base 1310 and/or may define one or more openings, voids, or spaces within the cages of FIGS. 13-16. For example, the cavities 1370 may extend into and/or through a side of the base 1310 (e.g., into and/or through the second side 1358, as shown in cage 1300 of FIGS. 13-14 and/or cage 1500 of FIGS. 15-16, etc.), a top of the base 1310 (e.g., into and/or through the top surface 1360, as shown in cage 1300 of FIGS. 13-14 and/or cage 1500 of FIGS. 15-16, etc.), and/or another suitable surface or component of the base 1310 (e.g., a bottom side or surface, etc.). In an exemplary embodiment, the cavities 1370 are defined by one or more components of the base 1310, and may be configured to receive one or more fluids and/or growth material (e.g., bone growth material, a graft, etc.), for example to facilitate healing, stability, and/or repair (e.g., of surrounding bone or adjacent bones, connected or surrounding tissue, and/or other anatomical features, etc.).

As discussed herein, the base 1310 of the cages of FIGS. 13-16 includes one or more strain gauges 1330, which may be the same as or similar to the strain gauge 230, 232 of FIGS. 1-7, and/or one or more compression apertures 1340, which may be the same as or similar to the compression apertures 240 of FIGS. 1-7, as described herein. For example, the base 1310 may include one strain gauge 1330 positioned at a surface of the base 1310 (e.g., a strain gauge positioned at surface that defines the cavity 1370, as shown in cage 1300 of FIG. 14).

In some embodiments, the base 1310 includes a strain gauge 1330 and a compression aperture 1340 positioned adjacent the strain gauge 1330 (e.g., as shown in cage 1300 of FIGS. 13-14, etc.). The compression aperture 1340 may extend through the base 1310 (e.g., a bottom of the base 1310, as shown in cage 1300 of FIG. 14, etc.) and/or into a portion of the base 1310 (e.g., into a side of the base 1310 and to a cavity of the base 1310, as shown in cage 1300 of FIG. 13, etc.). In other embodiments, the base 1310 includes a strain gauge 1330 without a compression aperture 1340 (e.g., as shown in cage 1500 of FIG. 16, etc.), for example due to the positioning and/or characteristics of the base 1310 where the strain gauge 1330 is positioned (e.g., at an expansion member, at a pivot member, etc.).

In some embodiments, the base 1310 includes a plurality of strain gauges 1330 positioned at one or more surfaces of the base 1310, and/or a plurality of compression apertures 1340 (e.g., a plurality of strain gauges and compression apertures positioned at a top surface, as shown in cage 1300 of FIG. 13, etc.). As discussed herein, the strain gauges 1330 and/or compression apertures 1340 may also be positioned relative to one or more components and/or portions the cages of FIGS. 13-16 (e.g., relative to the base 1310, etc.). For example, the strain gauges 1330 may be positioned at one or more edges or sides of the cage (e.g., a first side and/or a second side of the cage, as shown in the cage 1300 of FIG. 13, etc.), which may or may not be positioned relative to one or more cavities 1370 (e.g., between a side and a cavity the cage, as shown in the cage 1300 of FIG. 13, etc.). As discussed herein, the strain gauges 1330 may or may not be positioned adjacent one or more compression apertures 1340 (e.g., at an expandable member without a compression aperture, as shown in the cage 1500 of FIG. 16, etc.). Further, the strain gauges 1330 and/or compression apertures 1340 may also be positioned between and/or relative to one or more areas or regions of the cages (e.g., within a cavity of the cage, as shown in the cage 1300 of FIG. 14; within an area or at a component of expansion and/or contraction, as shown in cage 1500 of FIG. 16, etc.).

It should be understood that it is contemplated that the strain gauges 1330 and/or compression apertures 1340 may be or include any other suitable number of strain gauges and/or compression apertures (e.g., 3, 4, 5, 8, 10, 15, etc.), which may be otherwise positioned, oriented, and/or configured relative to one or more components of a cage (e.g., the cages of FIGS. 13-15). All such embodiments are contemplated herein.

According to an exemplary embodiment, and as described herein, the strain gauges 1330 and compression apertures 1340 of FIGS. 13-16 are configured to measure (e.g., determine, collect, calculate, compute, etc.) a value of one or more parameters (e.g., stress, strain, torque, pressure, temperature, movement, etc.) at and/or surrounding the cages of FIGS. 13-16. In an example embodiment, the strain gauges 1330 may be configured to determine (e.g., measure, collect, etc.) a value of one or more parameters (e.g., a stress, a strain, a temperature, a movement, etc.) of the cage itself. For example, the strain gauges 1330 and/or the compression apertures 1340 may be configured to collect stress, strain, and/or pressure data, for example to evaluate and/or assess conditions as the cage moves between different configurations (e.g., between a collapsed configuration, and an expanded configuration, etc.). In other embodiments, the strain gauges 1330 may be configured to determine a value of one or more parameters (e.g., pressure, temperature, etc.) associated with an anatomical feature surrounding the cage (e.g., a strain imparted on/by a bone coupled with the cage, a temperature of a surrounding tissue, a movement of an adjacent graft, etc.). For example, the strain gauges 1330 and/or the compression apertures 1340 may be configured to collect stress, strain, and/or pressure data, which may be useful in determining a state or effectiveness of a fusion event. As described herein, this information may be communicated (e.g., to a computing system, etc.), for example to facilitate monitoring, analysis, and/or evaluation of one or more characteristics associated with the devices and/or surrounding anatomical features.

In some embodiments, the compression apertures 1340 (or other compression apertures herein) can be filed with a softer material than that of a remaining structure of the cage (or other implant) (e.g., silicone) so as to prevent ingrowth of tissue into the compression apertures 1340 while still allowing the compression apertures 1340 to provide for measurable strain to be experienced at the strain gauges 1330. In other scenarios, tissue is expected or intended to grow into the compression apertures 1340 (or other compression apertures herein) and such growth and progress (e.g., toward fusion, grafting, soft-tissue healing, etc.) can be assessed using measurements from the strain gauges 1330 (or other strain gauges here).

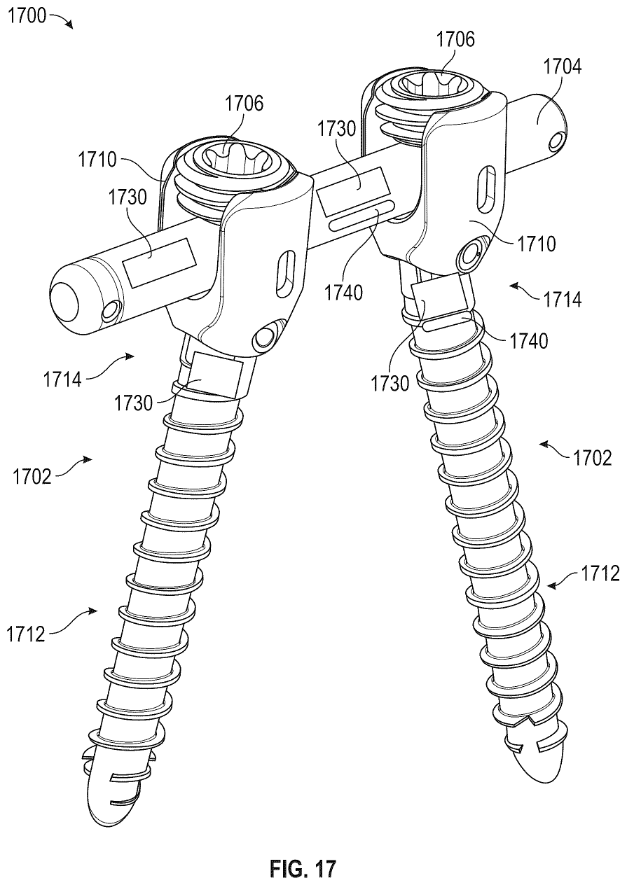

Referring now to FIG. 17, an implant system, a stabilization system, or a prosthetic system, shown as system 1700, is shown, according to an exemplary embodiment. In an exemplary embodiment, the system 1700 is a stabilization system, for example for stabilizing, fixating, and repairing various bones or bone structures (e.g., vertebrae, the spine, etc.). It should be understood that while system 1700 is described herein as being a vertebral stabilization system, it is contemplated that in other embodiments the system 1700 is another suitable system for use in a variety of other surgical procedures described herein (e.g., hip, shoulder, knee, hand, wrist, foot, ankle, vertebral, cranial, neurological, etc. applications) (e.g., the screws of FIG. 17 can be used for fixation of an acetabular cup implant or implant augment for a hip arthroplasty procedure, of a plate of FIGS. 8-11, of a tibial or femoral implant, etc.).

As shown, the system 1700 includes one or more anchors, shown as anchors 1702, a stabilizer, shown as a rod 1704, and one or more connectors, shown as caps 1706. In an exemplary embodiment, the anchors 1702 are bone screws; however, in other embodiments the anchors 1702 are another suitable anchor and/or stabilization component or device (e.g., bolt, rod, pin, barb, etc.). The rod 1704 is shown as a cylindrical beam or tube, which may be configured to couple one or more anchors 1702, for example to stabilize (e.g., secure, etc.) the anchors 1702 relative to one another.

Each anchor 1702 is shown to include a head region, shown as head 1710, a base or shaft region, shown as shaft 1712, and a neck 1714 between the head 1710 and the shaft 1712. The head 1710 is shown to include a receiver, cup, channel, or groove, for example to receive the rod 1704. The head 1710 may further include a cup or cap interface (e.g., a threaded interface, etc.), for example to receive and/or couple the cap 1706 (e.g., to secure the anchor 1702 relative to the rod 1704, etc.). The shaft 1712 is shown to include a tip and/or an exterior threading, which may be used to couple the anchor 1702 to one or more bones or bone structures. In an exemplary embodiment, each anchor 1702 is coupled with one or more bones or bone structures (e.g., adjacent vertebrate, for example via the threading of the shaft 1712, etc.), the rod 1704 is received by the head 1710 of each anchor 1702 (e.g., within the cup or channel, etc.), and the cap 1706 is secured to the head 1710, thereby coupling the rod 1704 to the head 1710 of each anchor 1702 to stabilize and/or secure the anchors 1702 and the one or more bones connected thereto.

As shown, the system 1700 includes one or more strain gauges, shown as strain gauges 1730, and one or more compression apertures, shown as compression apertures 1740. The strain gauges 1730 may be the same as or similar to the strain gauges 230, 232, and the compression apertures 1740 may be the same as or similar to the compression aperture 240, as described herein with reference to FIGS. 1-7. In this regard, the compression apertures 1740 and/or the strain gauges 1730 may be specifically positioned relative to one another, and/or relative to other components of the system 1700 of FIG. 17, as described herein.

In an exemplary embodiment, one or more of the anchors 1702 includes one or more strain gauges 1730. For example, the anchor 1702 may include a strain gauge 1730 positioned at the neck 1714. In some embodiments, the anchor 1702 also includes the compression aperture 1740, for example positioned adjacent the strain gauge 1730 (e.g., as shown in an anchor of FIG. 17). In other embodiments, the anchor 1702 does not include a compression aperture 1740, and only includes a strain gauge 1730 (e.g., as shown in an anchor of FIG. 17). In some embodiments, the anchors 1702 include a plurality of strain gauges 1730 and/or compression apertures 1740. For example, the anchors 1702 may include a plurality of strain gauges 1730 and/or compression apertures 1740 positioned throughout the neck 1714, the shaft 1712, and/or the head 1710 (e.g., at the cup or channel, at a base, etc.).

In an exemplary embodiment, the rod 1704 also includes one or more strain gauges 1730. For example, the rod 1704 may include a plurality of strain gauges 1730 positioned throughout a length of the rod 1704. In an exemplary embodiment, the rod 1704 may include a plurality of strain gauges 1730 that are spaced and/or positioned at predetermined intervals, for example such that the strain gauges 1730 are positioned between regions configured to receive the anchors 1702 (e.g., as shown in FIG. 17). In some embodiments, the rod 1704 also includes one or more compression apertures 1740, for example positioned adjacent the strain gauge 1730 (e.g., as shown in a region between the anchors in FIG. 17). The compression apertures 1740 may extend through and/or into the rod 1704. In other embodiments, the rod 1704 does not include compression apertures 1740 at each strain gauge 1730 (e.g., as shown at a strain gauge in FIG. 17), but rather may include compression apertures 1740 that are spaced and/or positioned at predetermined intervals (e.g., at every other strain gauge, at alternating strain gauges, etc.).

It should be understood that it is contemplated that the strain gauges 1730 and/or compression apertures 1740 may be or include any other suitable number of strain gauges and/or compression apertures (e.g., 3, 4, 5, 8, 10, 15, etc.), which may be otherwise positioned, oriented, and/or configured relative to one or more components of the system 1700 (e.g., the anchors 1702, the rod 1704, the caps 1706, etc.). All such embodiments are contemplated herein.

According to an exemplary embodiment, and as described herein, the strain gauges 1730 and compression apertures 1740 of the system 1700 are configured to measure (e.g., determine, collect, calculate, compute, etc.) a value of one or more parameters (e.g., stress, strain, torque, pressure, temperature, movement, etc.) at and/or surrounding one or more components of the system 1700. In an example embodiment, the strain gauges 1730 may be configured to determine (e.g., measure, collect, etc.) a value of one or more parameters (e.g., a stress, a strain, a temperature, a movement, etc.) of an anchor 1702 and/or the rod 1704 itself. For example, the strain gauges 1730 may be configured to determine a load (e.g., stress, strain, pressure, etc.) applied at an anchor or a series of anchors 1702, and/or a load distributed across the rod 1704. Further, the strain gauges 1730 may be configured to determine a value of one or more parameters (e.g., pressure, temperature, etc.) associated with an anatomical feature surrounding an anchor 1702, the rod 1704, and/or the cap 1706 (e.g., a strain imparted on/by a bone coupled with an anchor, a temperature of a surrounding tissue, a movement of an adjacent graft, etc.). For example, the strain gauges 1730 may be configured to determine a force or load imparted (e.g., on an anchor 1702, the rod 1704, etc.) by a muscle, tendon, and/or tissue coupled with the system 1700. As described herein, this information may be communicated (e.g., to a computing system, etc.), for example to facilitate monitoring, analysis, and/or evaluation of one or more characteristics associated with the system and/or surrounding anatomical features.

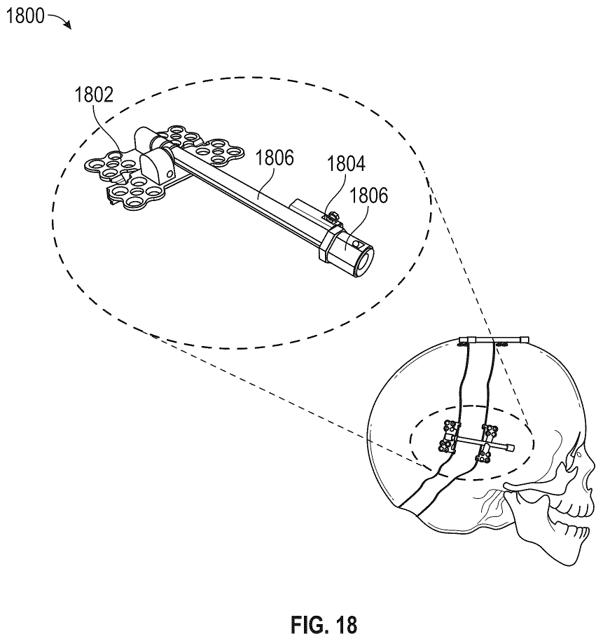

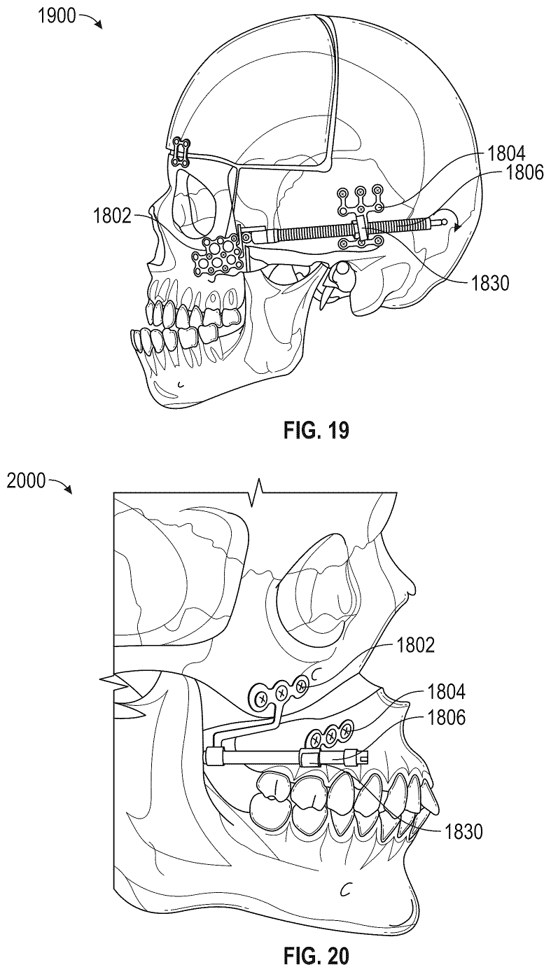

Referring now to FIGS. 18-20, a plurality of implants, implantable devices, and/or prosthetic devices are shown, according to various embodiments. In an exemplary embodiment, the devices of FIGS. 18-20 are distraction devices (e.g., osteogenesis distraction devices, etc.). For example, the distraction device may be a device for cranial vault or posterior cranial vault distraction, shown as device 1800 (e.g., as shown in FIG. 18), a device for frontofacial or monobloc frontofacial advancement, shown as device 1900 (e.g., as shown in FIG. 19), and/or a device for maxillary advancement, shown as device 2000 (e.g., as shown in FIG. 20).

It should be understood that while the devices of FIGS. 18-20 are described herein as being implants or devices for use in certain distraction procedures, it is contemplated that in other embodiments the devices of FIGS. 18-20 are implants or devices, which may be used in a variety of surgical (e.g., distraction, etc.) procedures. For example, the devices of FIGS. 18-20 may be used in procedures relating to posterior cranial vault distraction (e.g., to facilitate brain growth, etc.), frontofacial advancement (e.g. to facilitate repositioning of the forehead and/or midface, etc.), subcranial distraction (e.g., to facilitate normalization of proportions of the face, etc.) subcranial rotation distraction (e.g., to improve air flow and/or jaw position, etc.), maxillary advancement (e.g., to correct upper jaw displacement, etc.), midfacial advancement (e.g., to reposition the middle of the face, etc.), mandible distraction (e.g., for lower jaw repair, etc.), epiphyseal distraction (e.g., for limb lengthening, etc.), and/or any other suitable procedure or technique.

In an exemplary embodiment, each of the devices 1800, 1900, 2000 of FIGS. 18-20 may include one or more features and/or components, which may be similar and/or the same. For example, each of the devices 1800, 1900, 2000 are shown to include a first interface, connector, or anchor, shown as first anchor 1802, and a second interface, connector, or anchor, shown as second anchor 1804. The first anchor 1802 may be configured to couple (e.g., secure, attach, anchor, etc.) a first bone or bone structure, for example via one or more anchors (e.g., pins, bone screws, bolts, rods, etc.), as described herein. The second anchor 1804 may be configured to couple (e.g., secure, attach, anchor, etc.) a second bone or bone structure, for example via one or more anchors (e.g., pins, bone screws, bolts, rods, etc.).

As shown, the first anchor 1802 and the second anchor 1804 may be coupled via a controller, connector, driver, expander, or distractor, shown as connector 1806. The connector 1806 may be or include one or more drive members (e.g., a threaded drive shaft, etc.), which may be used to control a position, orientation, and/or configuration of the first anchor 1802 and/or the second anchor 1804. In this regard, the connector 1806 may be configured to be manipulated to control a configuration and/or position of the first anchor 1802 (e.g., and associated first bone) and the second anchor o1804 (e.g., and attached second bone, etc.). For example, the connector 1806 may be a threaded shaft, which when manipulated (e.g., rotated, etc.) may alter a distance between the first anchor 1802 (e.g., and associated first attached bone) and the second anchor 1804 (e.g., and associated second attached bone). In some embodiments, the connector 1806 is configured to be manipulated (e.g., rotated, etc.), for example to increase a distance between the first anchor 1802 and the second anchor 1804, for example to facilitate growth, healing, and/or repair in one or more spaces between the first anchor 1802 and the second anchor 1804 (e.g., in a space or cavity between the first and second bones, etc.).

As shown, the devices of FIGS. 18-20 also include one or more strain gauges, shown as strain gauges 1830, and one or more compression apertures, shown as compression apertures 1840. The strain gauges 1830 may be the same as or similar to the strain gauges 230, 232, and the compression apertures 1840 may be the same as or similar to the compression apertures 240, as described herein with reference to FIGS. 1-7. In this regard, the compression apertures 1840 and/or the strain gauges 1830 may be positioned relative to one another, and/or relative to other components of the devices of FIG. 18-20, as described herein.

In an exemplary embodiment, the devices of FIGS. 18-20 include one strain gauge 1830 positioned at a surface of an anchor. For example, the devices of FIGS. 18-20 may include a strain gauge 1830 positioned at an interface or connection point between the second anchor 1804 and the connector 1806 (e.g., as shown in FIGS. 18-20). In some embodiments, the devices of FIGS. 18-20 include one or more strain gauges 1830, for example positioned at a body or base of an anchor (e.g., a body or base of the first anchor 1802, the second anchor 1804, etc.). In some embodiments, the devices of FIGS. 18-20 further include one or more compression apertures 1840, for example positioned adjacent the strain gauges 1830 (e.g., at the body or base of the first anchor 1802, the second anchor 1804, etc.). In other embodiments, the devices of FIGS. 18-20 include one or more strain gauges 1830 without one or more compression apertures 1840 (e.g., as shown in FIGS. 18-20), for example as a result of the positioning of the strain gauges 1830 and/or the configuration of the devices (e.g., adjacent an opening, etc.).

In other embodiments, the devices of FIGS. 18-20 include a plurality of strain gauges 1830 and/or compression apertures 1840. For example, the devices may include a plurality of strain gauges 1830 and/or compression apertures 1840 positioned at a first connection point between an anchor and the connector (e.g., a connection point between the second anchor 1804 and the connector 1806, as shown in FIGS. 18-20) and a second connection point between an anchor and the connector (e.g., connection point between the first anchor 1802 and the connector 1806, etc.). In other embodiments, the devices of FIGS. 18-20 include one or more strain gauges 1830 and/or compression apertures 1840, which may be otherwise positioned at the first anchor 1802, the second anchor 1804, and/or the connector 1806 (e.g., at a central or middle portion, at an end portion, etc.). It should be understood that it is contemplated that the strain gauges 1830 and/or compression apertures 1840 may be or include any other suitable number of strain gauges and/or compression apertures (e.g., 3, 4, 5, 8, 10, 15, etc.), which may be otherwise positioned, oriented, and/or configured relative to one or more components of the devices of FIGS. 18-20. All such embodiments are contemplated herein.

According to an exemplary embodiment, and as described herein, the strain gauges 1830 and/or compression apertures 1840 of the devices of FIGS. 18-20 are configured to measure (e.g., determine, collect, calculate, compute, etc.) a value of one or more parameters (e.g., stress, strain, torque, pressure, temperature, movement, etc.) at and/or surrounding one or more components of the devices of FIGS. 18-20. In an example embodiment, the strain gauges 1830 are configured to determine (e.g., measure, collect, etc.) a value of one or more parameters (e.g., a stress, a strain, a temperature, a movement, etc.) of an anchor and/or a connector itself. For example, the strain gauges 1830 may be configured to determine a load (e.g., stress, strain, pressure, etc.) applied at an anchor or a series of anchors (e.g., the first anchor 1802, the second anchor 1804, etc.), and/or a load distributed across the connector 1806 (e.g., during a distraction, as a result of a distraction, etc.). Further, the strain gauges 1830 may be configured to determine a value of one or more parameters (e.g., pressure, temperature, etc.) associated with an anatomical feature surrounding an anchor (e.g., the first anchor 1802, the second anchor 1804, etc.) or the connector 1806 (e.g., a strain imparted on/by a bone coupled with an anchor, a temperature of a surrounding tissue, a movement of an adjacent graft, etc.). For example, the strain gauges 1830 may be configured to determine a force or load imparted (e.g., on an anchor or the connector, etc.) by a muscle, tendon, and/or tissue coupled with the devices of FIGS. 18-20, for example to evaluate growth, repair, and/or healing associated with the surrounding anatomical features. As described herein, this information may be communicated (e.g., to a computing system, etc.), for example to facilitate monitoring, analysis, and/or evaluation of one or more characteristics associated with the devices and/or surrounding anatomical features.

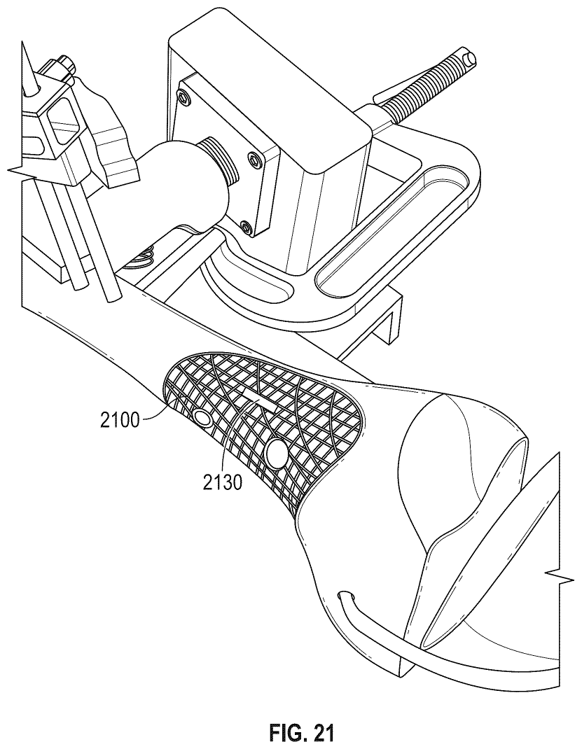

Referring now to FIG. 21, an implant, implantable device, and/or prosthetic device, shown as implant 2100, is shown, according to an exemplary embodiment. In an exemplary embodiment, the implant 2100 is an implant or prosthetic device, which may be used to repair or replace bone. For example, the implant 2100 may be used to repair or replace resected bone, such as surrounding a removed tumor or other diseased tissue.

As shown, the implant 2100 may include one or more strain gauges, shown as strain gauge 2130, which may be the same as or similar to the strain gauges 230, 232 described herein with reference to FIGS. 1-7. In some embodiments, the implant 2100 is a solid implant. In such instances, the implant 2100 may further include one or more compression apertures 2140, with may be the same as or similar to the compression aperture 240, as described herein with reference to FIGS. 1-7. In other embodiments, the implant 2100 includes a matrix or webbed structure, for example creating one or more spaces, voids, openings, and/or apertures in the implant 2100 (e.g., to receive fluid, growth material, etc.). In such embodiments, the implant 2100 may include one or more strain gauges 2130, for example without the one or more compression apertures.

According to an exemplary embodiment, and as described herein, the strain gauge 2130 of the implant 2100 is configured to measure (e.g., determine, collect, calculate, compute, etc.) a value of one or more parameters (e.g., stress, strain, torque, pressure, temperature, movement, etc.) at and/or surrounding the implant 2100. For example, the strain gauge 2130 may be configured to determine (e.g., measure, collect, etc.) a value of one or more parameters (e.g., a stress, a strain, a temperature, a movement, etc.) of the implant 2100 itself (e.g., during activity, movement, healing or repair, etc.). Further, the strain gauge 2130 may be configured to determine a value of one or more parameters (e.g., pressure, temperature, etc.) associated with an anatomical feature surrounding the implant 2130 (e.g., a strain imparted on/by a bone coupled with the implant, a temperature of a surrounding tissue, a movement of an adjacent graft, etc.). As described herein, this information may be communicated (e.g., to a computing system, etc.), for example to facilitate monitoring, analysis, and/or evaluation of one or more characteristics associated with the implant and/or surrounding anatomical features.

Referring now to FIG. 22, a system 2200 for collecting, obtaining, determining, communicating, analyzing, and/or evaluating information (e.g., data, etc.) is shown, according to an exemplary embodiment. In an exemplary embodiment, the system 2200 is or includes one or more components of the surgical system 100 of FIG. 1, and/or the devices (e.g., distraction lever, implants, plates, cages, systems, etc.) described with reference to FIGS. 1-20.

As shown, the system 2200 includes a system 2202, a remote device 2204, and a device 2206. In an exemplary embodiment, the system 2202 is or includes one or more components of the surgical system 100 of FIG. 1. For example, the system 2202 may be or include the robotic system 102, the computing system 104, and/or the other components, devices, and/or systems described herein. The remote device 2204 may be a computing device, a user device (e.g., a phone, tablet, computing station, memory device, wearable device, etc.), and/or another suitable computing and/or storage device. As shown, the system 2202, the remote device 2204, and/or the device 2206 may be communicably coupled. For example, the system 2202, the remote device 2204, and/or the device 2206 via one or more communications interfaces, networks, wired and/or wireless connections, and/or any other suitable communications systems and/or components, for example to communicate information between the system 2202, the remote device 2204, and/or the device 2206.

In an exemplary embodiment, the device 2206 is or includes one or more of the devices described herein. For example, the device 2206 may be the distraction lever 200 of FIGS. 1-7, the plates of FIGS. 8-11, the implant of FIG. 12, the cages of FIGS. 13-16, the devices of the system of FIG. 17, the distraction devices or devices of FIGS. 18-20, and/or the implant of FIG. 21. All such embodiments are contemplated herein.

As shown, the device 2206 includes a strain gauge 2230. According to an exemplary embodiment, the strain gauge 2230 is the same as or similar to any and/or all of the strain gauges described herein. For example, the strain gauge 2230 may be the strain gauge 230 of FIGS. 1-7, the strain gauge 830 of FIGS. 8-11, the strain gauge 1230 of FIG. 12, the strain gauge 1330 of FIGS. 13-16, the strain gauge 1730 of FIG. 17, the strain gauge 1830 of FIGS. 18-20, and/or the strain gauge 2130 of FIG. 21. All such embodiments are contemplated herein.

The strain gauge 2230 is shown to include a communications interface or device (e.g., network device, network interface, etc.), shown as antenna 2232, and a storage or memory device (e.g., one or more memory devices, computer-readable instructions stored on one or more memory devices), shown as memory 2234. According to an exemplary embodiment, and as discussed herein, the antenna 2232 may be configured to communicate information from the device 2206 (e.g., the strain gauge 2230, etc.) to one or more components of the system 2200 (e.g., the system 2202, the remote device 2204, etc.), for example via wireless or wired communications. For example, the antenna 2232 may be configured to have a current induced therein by an oscillating electric field from the system 2202 or remote device 2204 (e.g., via operating as a radio-frequency identification tag or near-field communication chip, etc.) thereby obtaining power for use by the strain gauge 2230, memory 2234, antenna 2232, etc. in collecting and transmitting measurements of the strain gauge 2230.

In an exemplary embodiment, the memory 2234 is configured to receive, store, and/or provide information, for example associated with the device 2206. For example, and as described herein, the strain gauge 2230 may be configured to measure (e.g., determine, collect, calculate, compute, etc.) a value of one or more parameters (e.g., stress, strain, torque, pressure, temperature, movement, etc.) at and/or surrounding the device 2206. The memory 2234 may be configured to receive and/or store the information gathered or obtained by the strain gauge 2230. In an exemplary embodiment, the memory 2234 may also be configured to communicate the information (e.g., stored information, gathered information, etc.) to the antenna 2232, for example for further communication to the one or more components of the system 2220, as described herein.

In an exemplary embodiment, the device 2206 (e.g., the strain gage 2230) is configured to obtain information, store information, and/or communicate information in real-time and/or near real-time. For example, the strain gauge 2230 may obtain information associated with one or more parameters at and/or surrounding the device 2206, the memory 2234 may store the information, and/or the antenna 2232 may communicate the information to the one or more components of the system 2220 (e.g., the system 2202, the remote device 2204, etc.) in real-time or near real-time, for example for subsequent analysis and/or evaluation. For example, a patient may wear the remote device 2204 external to the patient's body at a location proximate the device 2206, and the remote device 2204 can provide an electric filed sufficient to induce current in the antenna 2232 and cause measurements of the strain gauge 2230 to stream to the remote device 2204 in real time.

In some embodiments, the device 2206 (e.g., the strain gauge 2230) is configured to obtain and/or store information, and communicate information at predetermined intervals and/or in response to one or more instructions or operations. For example, the strain gauge 2230 may obtain information associated with one or more parameters at and/or surrounding the device 2206, and the memory 2234 may store the information. In some embodiments, in response to the device 2206 (e.g., the strain gauge 2230, etc.) receiving an instruction or call (e.g., a retrieval instruction or command or signal inducing a current from the system 2202, the remote device 2204, etc.), the memory 2234 may communicate the stored information to the antenna 2232, which may be communicated the one or more components of the system 2220 (e.g., the system 2202, the remote device 2204, etc.) for example for subsequent analysis and/or evaluation. In some embodiments, in response to the device 2206 (e.g., the strain gauge 2230, etc.) establishing a communications session or communications link with another device (e.g., a short-range wireless communications or near field communication session, for example with a device of the system 2202 or remote device 2204, etc.), the memory 2234 may communicate the stored information to the antenna 2232, which may be communicated the one or more components of the system 2220 (e.g., the system 2202, the remote device 2204, etc.) for example for subsequent analysis and/or evaluation.

In this regard, it is contemplated that the strain gauge 2230 may be configured to determine (e.g., obtain, collect, gather, measure, etc.) information and/or data associated with one or more parameters at and/or surrounding the device 2206, which may be communicated to one or more components of the system 2200 continuously and/or in real-time or near real-time, and/or stored (e.g., in the memory 2234, etc.) and communicated to one or more components of the system 2200 at predetermined intervals and/or in response to one or more instructions or operations. All such embodiments are contemplated herein.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single-or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general-purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general-purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above.

It is important to note that any element disclosed in one embodiment may be incorporated or utilized with any other embodiment disclosed herein. For example, the process of performing a joint distraction procedure of the exemplary embodiment described in at least FIG. 6 may be incorporated in the process of performing a gap balancing procedure of the exemplary embodiment described in at least FIG. 7. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A joint distraction lever, comprising:
a lever body having a distal end and a proximal end opposite the distal end, the distal end having a handle portion and the proximal end having a working portion configured to be positioned between a first bone and a second bone of a joint; and
a strain gauge integrated into the lever body and positioned between the handle portion and the working portion, the strain gauge configured to measure a strain on the lever body indicative of a distraction force applied at the distal end during a distraction procedure.

2. The joint distraction lever of claim 1, where the strain gauge is integrated into the lever body via a photodeposition process.

3. The joint distraction lever of claim 1, where the lever body further comprises a top surface and a bottom surface opposite the top surface, and a first lateral side surface and a second lateral side surface opposite the first lateral side surface, the first lateral side surface and the second lateral side surface extending from the handle portion to the working portion, and where the joint distraction lever further comprises a compression aperture configured to permit at least a portion of the lever body to deform in response to the distraction force applied at the distal end.

4. The joint distraction lever of claim 3, wherein the strain gauge is integrated into the top surface of the lever body.

5. The joint distraction lever of claim 3, wherein the compression aperture is positioned between the handle portion and the working portion, and extends through the lever body from the first lateral side surface to the second lateral side surface, where the strain gauge is integrated with the top surface of the lever body above the compression aperture.

6. The joint distraction lever of claim 3, wherein the compression aperture extends through the lever body from the first lateral side surface to the second lateral side surface and has a cross-sectional shape that is substantially rectangular and includes circular features at each corner.

7. The joint distraction lever of claim 1, wherein the strain gauge is a first strain gauge of a plurality of strain gauges including a second strain gauge.

8. The joint distraction lever of claim 7, wherein the first strain gauge is integrated with and extends into a top surface of the lever body, and the second strain gauge is integrated with and extends into a bottom surface of the lever body opposite the first strain gauge.

9. The joint distraction lever of claim 8, wherein the first strain gauge and the second strain gauge are integrated with the lever body via photodeposition processes.

10. The joint distraction lever of claim 1, wherein a surface of the lever body includes a dielectric substrate, and wherein the strain gauge is integrated into the surface of the lever body on the dielectric substrate via a photodeposition process.

11. The joint distraction lever of claim 10, wherein the dielectric substrate includes an oxide compound configured to bond a conductive material of the surface of the lever body, wherein the dielectric substrate is provided on the surface via a photodeposition process.

12. A surgical system, comprising:
a joint distraction lever, comprising:
a lever body having a distal end and a proximal end opposite the distal end, the distal end having a handle portion and the proximal end having a working portion configured to be positioned between a first bone and a second bone of a joint;
a strain gauge integrated into the lever body and positioned between the handle portion and the working portion, the strain gauge configured to measure a strain on the lever body indicative of a distraction force applied at the distal end during a distraction procedure;
a computing system configured to provide a surgical plan; and
a robotic device coupled with the joint distraction lever, the robotic device configured to apply a force to the handle portion in accordance with the surgical plan to cause the working portion to apply the distraction force.

13. The surgical system of claim 12, where the strain gauge is integrated with the lever body via a photodeposition process.

14. The surgical system of claim 12, where the lever body further comprises a top surface and a bottom surface opposite the top surface, and a first lateral side surface and a second lateral side surface opposite the first lateral side surface, the first lateral side surface and the second lateral side surface extending from the handle portion to the working portion, and where the joint distraction lever further comprises a compression aperture configured to permit at least a portion of the lever body to bias in response to the distraction force applied at the distal end.

15. The surgical system of claim 14, wherein the strain gauge is integrated with and extends into the top surface of the lever body.

16. The joint distraction lever of claim 14, wherein the compression aperture is positioned between the handle portion and the working portion, and extends through the lever body from the first lateral side surface to the second lateral side surface, where the strain gauge is integrated with the top surface of the lever body above the compression aperture.

17. The joint distraction lever of claim 14, wherein the compression aperture extends through the lever body from the first lateral side surface to the second lateral side surface and has a cross-sectional shape that is substantially rectangular and includes circular features at each corner.

18. A method of applying a distraction force during a distraction procedure, comprising:
providing a joint distraction lever, comprising:
a lever body having a distal end and a proximal end opposite the distal end, the distal end having a handle portion and the proximal end having a working portion configured to be positioned between a first bone and a second bone of a joint;
a strain gauge integrated into the lever body and positioned between the handle portion and the working portion, the strain gauge configured to measure a strain on the lever body indicative of the distraction force applied at the distal end during the distraction procedure;
inserting the joint distraction lever into a space between the first bone and the second bone;
applying a force to the handle portion such that the working portion applies the distraction force to the joint;
measuring, via the strain gauge, a force value associated with the distraction force; and
outputting the measured force value associated with the distraction force.

19. The method of claim 18, where the strain gauge is integrated with the lever body via a photodeposition process.

20. The method of claim 18, wherein the lever body further comprises a top surface and a bottom surface opposite the top surface, and a first lateral side surface and a second lateral side surface opposite the first lateral side surface, the first lateral side surface and the second lateral side surface extending from the handle portion to the working portion, and where the joint distraction lever further comprises a compression aperture configured to permit at least a portion of the lever body to bias in response to the distraction force applied at the distal end.
